SEQUENCE LISTING (73 sequences)

SEQ ID NO: 1 - RNA, 19 nt, synthetic construct
  source 1..19: mol_type = other RNA
cgtccgtccgtgaaattcc

SEQ ID NO: 2 - RNA, 19 nt, synthetic construct
  source 1..19: mol_type = other RNA
ggaatttcacggacggacg

SEQ ID NO: 3 - RNA, 19 nt, synthetic construct
  source 1..19: mol_type = other RNA
gctttgagcggaacccgta

SEQ ID NO: 4 - RNA, 19 nt, synthetic construct
  source 1..19: mol_type = other RNA
tacgggttccgctcaaagc

SEQ ID NO: 5 - RNA, 19 nt, synthetic construct
  source 1..19: mol_type = other RNA
gggtccagatttggtttca

SEQ ID NO: 6 - RNA, 19 nt, synthetic construct
  source 1..19: mol_type = other RNA
tgaaaccaaatctggaccc

SEQ ID NO: 7 - RNA, 19 nt, synthetic construct
  source 1..19: mol_type = other RNA
ggtccagatttggtttcag

SEQ ID NO: 8 - RNA, 19 nt, synthetic construct
  source 1..19: mol_type = other RNA
ctgaaaccaaatctggacc

SEQ ID NO: 9 - RNA, 19 nt, synthetic construct
  source 1..19: mol_type = other RNA
tccagatttggtttcagaa

SEQ ID NO: 10 - RNA, 19 nt, synthetic construct
  source 1..19: mol_type = other RNA
ttctgaaaccaaatctgga

SEQ ID NO: 11 - RNA, 19 nt, synthetic construct
  source 1..19: mol_type = other RNA
ccagatttggtttcagaat

SEQ ID NO: 12 - RNA, 19 nt, synthetic construct
  source 1..19: mol_type = other RNA
attctgaaaccaaatctgg

SEQ ID NO: 13 - RNA, 19 nt, synthetic construct
  source 1..19: mol_type = other RNA
cagatttggtttcagaatg

SEQ ID NO: 14 - RNA, 19 nt, synthetic construct
  source 1..19: mol_type = other RNA
cattctgaaaccaaatctg

SEQ ID NO: 15 - RNA, 19 nt, synthetic construct
  source 1..19: mol_type = other RNA
agatttggtttcagaatga

SEQ ID NO: 16 - RNA, 19 nt, synthetic construct
  source 1..19: mol_type = other RNA
tcattctgaaaccaaatct

SEQ ID NO: 17 - RNA, 19 nt, synthetic construct
  source 1..19: mol_type = other RNA
gatttggtttcagaatgag

SEQ ID NO: 18 - RNA, 19 nt, synthetic construct
  source 1..19: mol_type = other RNA
ctcattctgaaaccaaatc

SEQ ID NO: 19 - RNA, 19 nt, synthetic construct
  source 1..19: mol_type = other RNA
ggattcagatctggtttca

SEQ ID NO: 20 - RNA, 19 nt, synthetic construct
  source 1..19: mol_type = other RNA
tgaaaccagatctgaatcc

SEQ ID NO: 21 - RNA, 19 nt, synthetic construct
  source 1..19: mol_type = other RNA
gatctggtttcagaatcga

SEQ ID NO: 22 - RNA, 19 nt, synthetic construct
  source 1..19: mol_type = other RNA
tcgattctgaaaccagatc

SEQ ID NO: 23 - RNA, 19 nt, synthetic construct
  source 1..19: mol_type = other RNA
atctggtttcagaatcgaa

SEQ ID NO: 24 - RNA, 19 nt, synthetic construct
  source 1..19: mol_type = other RNA
ttcgattctgaaaccagat

SEQ ID NO: 25 - RNA, 19 nt, synthetic construct
  source 1..19: mol_type = other RNA
aggcgcaacctctcctaga

SEQ ID NO: 26 - RNA, 19 nt, synthetic construct
  source 1..19: mol_type = other RNA
tctaggagaggttgcgcct

SEQ ID NO: 27 - RNA, 19 nt, synthetic construct
  source 1..19: mol_type = other RNA
cgaggaagaataccgggct

SEQ ID NO: 28 - RNA, 19 nt, synthetic construct
  source 1..19: mol_type = other RNA
agcccggtattcttcctcg

SEQ ID NO: 29 - RNA, 19 nt, synthetic construct
  source 1..19: mol_type = other RNA
ctctgctggaggagcttta

SEQ ID NO: 30 - RNA, 19 nt, synthetic construct
  source 1..19: mol_type = other RNA
taaagctcctccagcagag

SEQ ID NO: 31 - RNA, 19 nt, synthetic construct
  source 1..19: mol_type = other RNA
cgcggtgttctgcctttct

SEQ ID NO: 32 - RNA, 19 nt, synthetic construct
  source 1..19: mol_type = other RNA
agaatggcagttctccgcg

SEQ ID NO: 33 - RNA, 21 nt, synthetic construct
  modified_base 20..21: mod_base = OTHER; note = Thymine
  modified_base 1: mod_base = OTHER; note = Locked Nucleic Acid (LNA) base
  modified_base 2: mod_base = OTHER; note = 2'-O-Methyl (2'-OMe) modified RNA base
  modified_base 3: mod_base = OTHER; note = 2'-Flouro (2'-F) modified RNA base
  modified_base 4: mod_base = OTHER; note = 2'-O-Methyl (2'-OMe) modified RNA base
  modified_base 5: mod_base = OTHER; note = 2'-Flouro (2'-F) modified RNA base
  modified_base 6: mod_base = OTHER; note = 2'-O-Methyl (2'-OMe) modified RNA base
  modified_base 7: mod_base = OTHER; note = 2'-Flouro (2'-F) modified RNA base
  modified_base 8: mod_base = OTHER; note = 2'-O-Methyl (2'-OMe) modified RNA base
  modified_base 9: mod_base = OTHER; note = 2'-Flouro (2'-F) modified RNA base
  modified_base 10: mod_base = OTHER; note = 2'-O-Methyl (2'-OMe) modified RNA base
  modified_base 11: mod_base = OTHER; note = 2'-Flouro (2'-F) modified RNA base
  modified_base 12: mod_base = OTHER; note = 2'-O-Methyl (2'-OMe) modified RNA base
  modified_base 13: mod_base = OTHER; note = 2'-Flouro (2'-F) modified RNA base
  modified_base 14: mod_base = OTHER; note = 2'-O-Methyl (2'-OMe) modified RNA base
  modified_base 15: mod_base = OTHER; note = 2'-Flouro (2'-F) modified RNA base
  modified_base 16: mod_base = OTHER; note = 2'-O-Methyl (2'-OMe) modified RNA base
  modified_base 17: mod_base = OTHER; note = 2'-Flouro (2'-F) modified RNA base
  modified_base 18: mod_base = OTHER; note = 2'-O-Methyl (2'-OMe) modified RNA base
  modified_base 19: mod_base = OTHER; note = 2'-Flouro (2'-F) modified RNA base
  source 1..21: mol_type = other RNA
cgtccgtccgtgaaattcctt

SEQ ID NO: 34 - RNA, 21 nt, synthetic construct
  modified_base 20..21: mod_base = OTHER; note = thymine
  modified_base 1: mod_base = OTHER; note = 2'-O-Methyl (2'-OMe) modified RNA base
  modified_base 2: mod_base = OTHER; note = 2'-Flouro (2'-F) modified RNA base
  modified_base 3: mod_base = OTHER; note = 2'-O-Methyl (2'-OMe) modified RNA base
  modified_base 4: mod_base = OTHER; note = 2'-Flouro (2'-F) modified RNA base
  modified_base 5: mod_base = OTHER; note = 2'-O-Methyl (2'-OMe) modified RNA base
  modified_base 6: mod_base = OTHER; note = 2'-Flouro (2'-F) modified RNA base
  modified_base 7: mod_base = OTHER; note = 2'-O-Methyl (2'-OMe) modified RNA base
  modified_base 8: mod_base = OTHER; note = 2'-Flouro (2'-F) modified RNA base
  modified_base 9: mod_base = OTHER; note = 2'-O-Methyl (2'-OMe) modified RNA base
  modified_base 10: mod_base = OTHER; note = 2'-Flouro (2'-F) modified RNA base
  modified_base 11: mod_base = OTHER; note = 2'-O-Methyl (2'-OMe) modified RNA base
  modified_base 12: mod_base = OTHER; note = 2'-Flouro (2'-F) modified RNA base
  modified_base 13: mod_base = OTHER; note = 2'-O-Methyl (2'-OMe) modified RNA base
  modified_base 14: mod_base = OTHER; note = 2'-Flouro (2'-F) modified RNA base
  modified_base 15: mod_base = OTHER; note = 2'-O-Methyl (2'-OMe) modified RNA base
  modified_base 16: mod_base = OTHER; note = 2'-Flouro (2'-F) modified RNA base
  modified_base 17: mod_base = OTHER; note = 2'-O-Methyl (2'-OMe) modified RNA base
  modified_base 18: mod_base = OTHER; note = 2'-Flouro (2'-F) modified RNA base
  modified_base 19: mod_base = OTHER; note = 2'-O-Methyl (2'-OMe) modified RNA base
  source 1..21: mol_type = other RNA
ggaatttcacggacggacgtt

SEQ ID NO: 35 - RNA, 21 nt, synthetic construct
  modified_base 20..21: mod_base = OTHER; note = thymine
  modified_base 1: mod_base = OTHER; note = Locked Nucleic Acid (LNA) base
  modified_base 2: mod_base = OTHER; note = 2'-O-Methyl (2'-OMe) modified RNA base
  modified_base 3: mod_base = OTHER; note = 2'-Flouro (2'-F) modified RNA base
  modified_base 4: mod_base = OTHER; note = 2'-O-Methyl (2'-OMe) modified RNA base
  modified_base 5: mod_base = OTHER; note = 2'-Flouro (2'-F) modified RNA base
  modified_base 6: mod_base = OTHER; note = 2'-O-Methyl (2'-OMe) modified RNA base
  modified_base 7: mod_base = OTHER; note = 2'-Flouro (2'-F) modified RNA base
  modified_base 8: mod_base = OTHER; note = 2'-O-Methyl (2'-OMe) modified RNA base
  modified_base 9: mod_base = OTHER; note = 2'-Flouro (2'-F) modified RNA base
  modified_base 10: mod_base = OTHER; note = 2'-O-Methyl (2'-OMe) modified RNA base
  modified_base 11: mod_base = OTHER; note = 2'-Flouro (2'-F) modified RNA base
  modified_base 12: mod_base = OTHER; note = 2'-O-Methyl (2'-OMe) modified RNA base
  modified_base 13: mod_base = OTHER; note = 2'-Flouro (2'-F) modified RNA base
  modified_base 14: mod_base = OTHER; note = 2'-O-Methyl (2'-OMe) modified RNA base
  modified_base 15: mod_base = OTHER; note = 2'-Flouro (2'-F) modified RNA base
  modified_base 16: mod_base = OTHER; note = 2'-O-Methyl (2'-OMe) modified RNA base
  modified_base 17: mod_base = OTHER; note = 2'-Flouro (2'-F) modified RNA base
  modified_base 18: mod_base = OTHER; note = 2'-O-Methyl (2'-OMe) modified RNA base
  modified_base 19: mod_base = OTHER; note = 2'-Flouro (2'-F) modified RNA base
  source 1..21: mol_type = other RNA
gctttgagcggaacccgtatt

SEQ ID NO: 36 - RNA, 21 nt, synthetic construct
  modified_base 20..21: mod_base = OTHER; note = thymine
  modified_base 1: mod_base = OTHER; note = 2'-O-Methyl (2'-OMe) modified RNA base
  modified_base 2: mod_base = OTHER; note = 2'-Flouro (2'-F) modified RNA base
  modified_base 3: mod_base = OTHER; note = 2'-O-Methyl (2'-OMe) modified RNA base
  modified_base 4: mod_base = OTHER; note = 2'-Flouro (2'-F) modified RNA base
  modified_base 5: mod_base = OTHER; note = 2'-O-Methyl (2'-OMe) modified RNA base
  modified_base 6: mod_base = OTHER; note = 2'-Flouro (2'-F) modified RNA base
  modified_base 7: mod_base = OTHER; note = 2'-O-Methyl (2'-OMe) modified RNA base
  modified_base 8: mod_base = OTHER; note = 2'-Flouro (2'-F) modified RNA base
  modified_base 9: mod_base = OTHER; note = 2'-O-Methyl (2'-OMe) modified RNA base
  modified_base 10: mod_base = OTHER; note = 2'-Flouro (2'-F) modified RNA base
  modified_base 11: mod_base = OTHER; note = 2'-O-Methyl (2'-OMe) modified RNA base
  modified_base 12: mod_base = OTHER; note = 2'-Flouro (2'-F) modified RNA base
  modified_base 13: mod_base = OTHER; note = 2'-O-Methyl (2'-OMe) modified RNA base
  modified_base 14: mod_base = OTHER; note = 2'-Flouro (2'-F) modified RNA base
  modified_base 15: mod_base = OTHER; note = 2'-O-Methyl (2'-OMe) modified RNA base
  modified_base 16: mod_base = OTHER; note = 2'-Flouro (2'-F) modified RNA base
  modified_base 17: mod_base = OTHER; note = 2'-O-Methyl (2'-OMe) modified RNA base
  modified_base 18: mod_base = OTHER; note = 2'-Flouro (2'-F) modified RNA base
  modified_base 19: mod_base = OTHER; note = 2'-O-Methyl (2'-OMe) modified RNA base
  source 1..21: mol_type = other RNA
tacgggttccgctcaaagctt

SEQ ID NO: 37 - RNA, 21 nt, synthetic construct
  modified_base 20..21: mod_base = OTHER; note = thymine
  modified_base 1: mod_base = OTHER; note = Locked Nucleic Acid (LNA) base
  modified_base 2: mod_base = OTHER; note = 2'-O-Methyl (2'-OMe) modified RNA base
  modified_base 3: mod_base = OTHER; note = 2'-Flouro (2'-F) modified RNA base
  modified_base 4: mod_base = OTHER; note = 2'-O-Methyl (2'-OMe) modified RNA base
  modified_base 5: mod_base = OTHER; note = 2'-Flouro (2'-F) modified RNA base
  modified_base 6: mod_base = OTHER; note = 2'-O-Methyl (2'-OMe) modified RNA base
  modified_base 7: mod_base = OTHER; note = 2'-Flouro (2'-F) modified RNA base
  modified_base 8: mod_base = OTHER; note = 2'-O-Methyl (2'-OMe) modified RNA base
  modified_base 9: mod_base = OTHER; note = 2'-Flouro (2'-F) modified RNA base
  modified_base 10: mod_base = OTHER; note = 2'-O-Methyl (2'-OMe) modified RNA base
  modified_base 11: mod_base = OTHER; note = 2'-Flouro (2'-F) modified RNA base
  modified_base 12: mod_base = OTHER; note = 2'-O-Methyl (2'-OMe) modified RNA base
  modified_base 13: mod_base = OTHER; note = 2'-Flouro (2'-F) modified RNA base
  modified_base 14: mod_base = OTHER; note = 2'-O-Methyl (2'-OMe) modified RNA base
  modified_base 15: mod_base = OTHER; note = 2'-Flouro (2'-F) modified RNA base
  modified_base 16: mod_base = OTHER; note = 2'-O-Methyl (2'-OMe) modified RNA base
  modified_base 17: mod_base = OTHER; note = 2'-Flouro (2'-F) modified RNA base
  modified_base 18: mod_base = OTHER; note = 2'-O-Methyl (2'-OMe) modified RNA base
  modified_base 19: mod_base = OTHER; note = 2'-Flouro (2'-F) modified RNA base
  source 1..21: mol_type = other RNA
gggtccagatttggtttcatt

SEQ ID NO: 38 - RNA, 21 nt, synthetic construct
  modified_base 20..21: mod_base = OTHER; note = thymine
  modified_base 1: mod_base = OTHER; note = 2'-O-Methyl (2'-OMe) modified RNA base
  modified_base 2: mod_base = OTHER; note = 2'-Flouro (2'-F) modified RNA base
  modified_base 3: mod_base = OTHER; note = 2'-O-Methyl (2'-OMe) modified RNA base
  modified_base 4: mod_base = OTHER; note = 2'-Flouro (2'-F) modified RNA base
  modified_base 5: mod_base = OTHER; note = 2'-O-Methyl (2'-OMe) modified RNA base
  modified_base 6: mod_base = OTHER; note = 2'-Flouro (2'-F) modified RNA base
  modified_base 7: mod_base = OTHER; note = 2'-O-Methyl (2'-OMe) modified RNA base
  modified_base 8: mod_base = OTHER; note = 2'-Flouro (2'-F) modified RNA base
  modified_base 9: mod_base = OTHER; note = 2'-O-Methyl (2'-OMe) modified RNA base
  modified_base 10: mod_base = OTHER; note = 2'-Flouro (2'-F) modified RNA base
  modified_base 11: mod_base = OTHER; note = 2'-O-Methyl (2'-OMe) modified RNA base
  modified_base 12: mod_base = OTHER; note = 2'-Flouro (2'-F) modified RNA base
  modified_base 13: mod_base = OTHER; note = 2'-O-Methyl (2'-OMe) modified RNA base
  modified_base 14: mod_base = OTHER; note = 2'-Flouro (2'-F) modified RNA base
  modified_base 15: mod_base = OTHER; note = 2'-O-Methyl (2'-OMe) modified RNA base
  modified_base 16: mod_base = OTHER; note = 2'-Flouro (2'-F) modified RNA base
  modified_base 17: mod_base = OTHER; note = 2'-O-Methyl (2'-OMe) modified RNA base
  modified_base 18: mod_base = OTHER; note = 2'-Flouro (2'-F) modified RNA base
  modified_base 19: mod_base = OTHER; note = 2'-O-Methyl (2'-OMe) modified RNA base
  source 1..21: mol_type = other RNA
tgaaaccaaatctggaccctt

SEQ ID NO: 39 - RNA, 21 nt, synthetic construct
  modified_base 20..21: mod_base = OTHER; note = thymine
  modified_base 1: mod_base = OTHER; note = 2'-Flouro (2'-F) modified RNA base
  modified_base 2: mod_base = OTHER; note = 2'-O-Methyl (2'-OMe) modified RNA base
  modified_base 3: mod_base = OTHER; note = 2'-Flouro (2'-F) modified RNA base
  modified_base 4: mod_base = OTHER; note = 2'-O-Methyl (2'-OMe) modified RNA base
  modified_base 5: mod_base = OTHER; note = 2'-Flouro (2'-F) modified RNA base
  modified_base 6: mod_base = OTHER; note = 2'-O-Methyl (2'-OMe) modified RNA base
  modified_base 7: mod_base = OTHER; note = 2'-Flouro (2'-F) modified RNA base
  modified_base 8: mod_base = OTHER; note = 2'-O-Methyl (2'-OMe) modified RNA base
  modified_base 9: mod_base = OTHER; note = 2'-Flouro (2'-F) modified RNA base
  modified_base 10: mod_base = OTHER; note = 2'-O-Methyl (2'-OMe) modified RNA base
  modified_base 11: mod_base = OTHER; note = 2'-Flouro (2'-F) modified RNA base
  modified_base 12: mod_base = OTHER; note = 2'-O-Methyl (2'-OMe) modified RNA base
  modified_base 13: mod_base = OTHER; note = 2'-Flouro (2'-F) modified RNA base
  modified_base 14: mod_base = OTHER; note = 2'-O-Methyl (2'-OMe) modified RNA base
  modified_base 15: mod_base = OTHER; note = 2'-Flouro (2'-F) modified RNA base
  modified_base 16: mod_base = OTHER; note = 2'-O-Methyl (2'-OMe) modified RNA base
  modified_base 17: mod_base = OTHER; note = 2'-Flouro (2'-F) modified RNA base
  modified_base 18: mod_base = OTHER; note = 2'-O-Methyl (2'-OMe) modified RNA base
  modified_base 19: mod_base = OTHER; note = 2'-Flouro (2'-F) modified RNA base
  source 1..21: mol_type = other RNA
ggtccagatttggtttcagtt

SEQ ID NO: 40 - RNA, 21 nt, synthetic construct
  modified_base 20..21: mod_base = OTHER; note = thymine
  modified_base 1: mod_base = OTHER; note = 2'-O-Methyl (2'-OMe) modified RNA base
  modified_base 2: mod_base = OTHER; note = 2'-Flouro (2'-F) modified RNA base
  modified_base 3: mod_base = OTHER; note = 2'-O-Methyl (2'-OMe) modified RNA base
  modified_base 4: mod_base = OTHER; note = 2'-Flouro (2'-F) modified RNA base
  modified_base 5: mod_base = OTHER; note = 2'-O-Methyl (2'-OMe) modified RNA base
  modified_base 6: mod_base = OTHER; note = 2'-Flouro (2'-F) modified RNA base
  modified_base 7: mod_base = OTHER; note = 2'-O-Methyl (2'-OMe) modified RNA base
  modified_base 8: mod_base = OTHER; note = 2'-Flouro (2'-F) modified RNA base
  modified_base 9: mod_base = OTHER; note = 2'-O-Methyl (2'-OMe) modified RNA base
  modified_base 10: mod_base = OTHER; note = 2'-Flouro (2'-F) modified RNA base
  modified_base 11: mod_base = OTHER; note = 2'-O-Methyl (2'-OMe) modified RNA base
  modified_base 12: mod_base = OTHER; note = 2'-Flouro (2'-F) modified RNA base
  modified_base 13: mod_base = OTHER; note = 2'-O-Methyl (2'-OMe) modified RNA base
  modified_base 14: mod_base = OTHER; note = 2'-Flouro (2'-F) modified RNA base
  modified_base 15: mod_base = OTHER; note = 2'-O-Methyl (2'-OMe) modified RNA base
  modified_base 16: mod_base = OTHER; note = 2'-Flouro (2'-F) modified RNA base
  modified_base 17: mod_base = OTHER; note = 2'-O-Methyl (2'-OMe) modified RNA base
  modified_base 18: mod_base = OTHER; note = 2'-Flouro (2'-F) modified RNA base
  modified_base 19: mod_base = OTHER; note = 2'-O-Methyl (2'-OMe) modified RNA base
  source 1..21: mol_type = other RNA
ctgaaaccaaatctggacctt

SEQ ID NO: 41 - RNA, 21 nt, synthetic construct
  modified_base 20..21: mod_base = OTHER; note = thymine
  modified_base 1: mod_base = OTHER; note = Locked Nucleic Acid (LNA) base
  modified_base 2: mod_base = OTHER; note = 2'-O-Methyl (2'-OMe) modified RNA base
  modified_base 3: mod_base = OTHER; note = 2'-Flouro (2'-F) modified RNA base
  modified_base 4: mod_base = OTHER; note = 2'-O-Methyl (2'-OMe) modified RNA base
  modified_base 5: mod_base = OTHER; note = 2'-Flouro (2'-F) modified RNA base
  modified_base 6: mod_base = OTHER; note = 2'-O-Methyl (2'-OMe) modified RNA base
  modified_base 7: mod_base = OTHER; note = 2'-Flouro (2'-F) modified RNA base
  modified_base 8: mod_base = OTHER; note = 2'-O-Methyl (2'-OMe) modified RNA base
  modified_base 9: mod_base = OTHER; note = 2'-Flouro (2'-F) modified RNA base
  modified_base 10: mod_base = OTHER; note = 2'-O-Methyl (2'-OMe) modified RNA base
  modified_base 11: mod_base = OTHER; note = 2'-Flouro (2'-F) modified RNA base
  modified_base 12: mod_base = OTHER; note = 2'-O-Methyl (2'-OMe) modified RNA base
  modified_base 13: mod_base = OTHER; note = 2'-Flouro (2'-F) modified RNA base
  modified_base 14: mod_base = OTHER; note = 2'-O-Methyl (2'-OMe) modified RNA base
  modified_base 15: mod_base = OTHER; note = 2'-Flouro (2'-F) modified RNA base
  modified_base 16: mod_base = OTHER; note = 2'-O-Methyl (2'-OMe) modified RNA base
  modified_base 17: mod_base = OTHER; note = 2'-Flouro (2'-F) modified RNA base
  modified_base 18: mod_base = OTHER; note = 2'-O-Methyl (2'-OMe) modified RNA base
  modified_base 19: mod_base = OTHER; note = 2'-Flouro (2'-F) modified RNA base
  source 1..21: mol_type = other RNA
ggtccagatttggtttcagtt

SEQ ID NO: 42 - RNA, 21 nt, synthetic construct
  modified_base 20..21: mod_base = OTHER; note = thymine
  modified_base 1: mod_base = OTHER; note = Locked Nucleic Acid (LNA) base
  modified_base 2: mod_base = OTHER; note = 2'-O-Methyl (2'-OMe) modified RNA base
  modified_base 3: mod_base = OTHER; note = 2'-Flouro (2'-F) modified RNA base
  modified_base 4: mod_base = OTHER; note = 2'-O-Methyl (2'-OMe) modified RNA base
  modified_base 5: mod_base = OTHER; note = 2'-Flouro (2'-F) modified RNA base
  modified_base 6: mod_base = OTHER; note = 2'-O-Methyl (2'-OMe) modified RNA base
  modified_base 7: mod_base = OTHER; note = 2'-Flouro (2'-F) modified RNA base
  modified_base 8: mod_base = OTHER; note = 2'-O-Methyl (2'-OMe) modified RNA base
  modified_base 9: mod_base = OTHER; note = 2'-Flouro (2'-F) modified RNA base
  modified_base 10: mod_base = OTHER; note = 2'-O-Methyl (2'-OMe) modified RNA base
  modified_base 11: mod_base = OTHER; note = 2'-Flouro (2'-F) modified RNA base
  modified_base 12: mod_base = OTHER; note = 2'-O-Methyl (2'-OMe) modified RNA base
  modified_base 13: mod_base = OTHER; note = 2'-Flouro (2'-F) modified RNA base
  modified_base 14: mod_base = OTHER; note = 2'-O-Methyl (2'-OMe) modified RNA base
  modified_base 15: mod_base = OTHER; note = 2'-Flouro (2'-F) modified RNA base
  modified_base 16: mod_base = OTHER; note = 2'-O-Methyl (2'-OMe) modified RNA base
  modified_base 17: mod_base = OTHER; note = 2'-Flouro (2'-F) modified RNA base
  modified_base 18: mod_base = OTHER; note = 2'-O-Methyl (2'-OMe) modified RNA base
  modified_base 19: mod_base = OTHER; note = 2'-Flouro (2'-F) modified RNA base
  source 1..21: mol_type = other RNA
tccagatttggtttcagaatt

SEQ ID NO: 43 - RNA, 21 nt, synthetic construct
  modified_base 20..21: mod_base = OTHER; note = thymine
  modified_base 1: mod_base = OTHER; note = 2'-O-Methyl (2'-OMe) modified RNA base
  modified_base 2: mod_base = OTHER; note = 2'-Flouro (2'-F) modified RNA base
  modified_base 3: mod_base = OTHER; note = 2'-O-Methyl (2'-OMe) modified RNA base
  modified_base 4: mod_base = OTHER; note = 2'-Flouro (2'-F) modified RNA base
  modified_base 5: mod_base = OTHER; note = 2'-O-Methyl (2'-OMe) modified RNA base
  modified_base 6: mod_base = OTHER; note = 2'-Flouro (2'-F) modified RNA base
  modified_base 7: mod_base = OTHER; note = 2'-O-Methyl (2'-OMe) modified RNA base
  modified_base 8: mod_base = OTHER; note = 2'-Flouro (2'-F) modified RNA base
  modified_base 9: mod_base = OTHER; note = 2'-O-Methyl (2'-OMe) modified RNA base
  modified_base 10: mod_base = OTHER; note = 2'-Flouro (2'-F) modified RNA base
  modified_base 11: mod_base = OTHER; note = 2'-O-Methyl (2'-OMe) modified RNA base
  modified_base 12: mod_base = OTHER; note = 2'-Flouro (2'-F) modified RNA base
  modified_base 13: mod_base = OTHER; note = 2'-O-Methyl (2'-OMe) modified RNA base
  modified_base 14: mod_base = OTHER; note = 2'-Flouro (2'-F) modified RNA base
  modified_base 15: mod_base = OTHER; note = 2'-O-Methyl (2'-OMe) modified RNA base
  modified_base 16: mod_base = OTHER; note = 2'-Flouro (2'-F) modified RNA base
  modified_base 17: mod_base = OTHER; note = 2'-O-Methyl (2'-OMe) modified RNA base
  modified_base 18: mod_base = OTHER; note = 2'-Flouro (2'-F) modified RNA base
  modified_base 19: mod_base = OTHER; note = 2'-O-Methyl (2'-OMe) modified RNA base
  source 1..21: mol_type = other RNA
ttctgaaaccaaatctggatt

SEQ ID NO: 44 - RNA, 21 nt, synthetic construct
  modified_base 20..21: mod_base = OTHER; note = thymine
  modified_base 1: mod_base = OTHER; note = Locked Nucleic Acid (LNA) base
  modified_base 2: mod_base = OTHER; note = 2'-O-Methyl (2'-OMe) modified RNA base
  modified_base 3: mod_base = OTHER; note = 2'-Flouro (2'-F) modified RNA base
  modified_base 4: mod_base = OTHER; note = 2'-O-Methyl (2'-OMe) modified RNA base
  modified_base 5: mod_base = OTHER; note = 2'-Flouro (2'-F) modified RNA base
  modified_base 6: mod_base = OTHER; note = 2'-O-Methyl (2'-OMe) modified RNA base
  modified_base 7: mod_base = OTHER; note = 2'-Flouro (2'-F) modified RNA base
  modified_base 8: mod_base = OTHER; note = 2'-O-Methyl (2'-OMe) modified RNA base
  modified_base 9: mod_base = OTHER; note = 2'-Flouro (2'-F) modified RNA base
  modified_base 10: mod_base = OTHER; note = 2'-O-Methyl (2'-OMe) modified RNA base
  modified_base 11: mod_base = OTHER; note = 2'-Flouro (2'-F) modified RNA base
  modified_base 12: mod_base = OTHER; note = 2'-O-Methyl (2'-OMe) modified RNA base
  modified_base 13: mod_base = OTHER; note = 2'-Flouro (2'-F) modified RNA base
  modified_base 14: mod_base = OTHER; note = 2'-O-Methyl (2'-OMe) modified RNA base
  modified_base 15: mod_base = OTHER; note = 2'-Flouro (2'-F) modified RNA base
  modified_base 16: mod_base = OTHER; note = 2'-O-Methyl (2'-OMe) modified RNA base
  modified_base 17: mod_base = OTHER; note = 2'-Flouro (2'-F) modified RNA base
  modified_base 18: mod_base = OTHER; note = 2'-O-Methyl (2'-OMe) modified RNA base
  modified_base 19: mod_base = OTHER; note = 2'-Flouro (2'-F) modified RNA base
  source 1..21: mol_type = other RNA
ccagatttggtttcagaattt

SEQ ID NO: 45 - RNA, 21 nt, synthetic construct
  modified_base 20..21: mod_base = OTHER; note = thymine
  modified_base 1: mod_base = OTHER; note = 2'-O-Methyl (2'-OMe) modified RNA base
  modified_base 2: mod_base = OTHER; note = 2'-Flouro (2'-F) modified RNA base
  modified_base 3: mod_base = OTHER; note = 2'-O-Methyl (2'-OMe) modified RNA base
  modified_base 4: mod_base = OTHER; note = 2'-Flouro (2'-F) modified RNA base
  modified_base 5: mod_base = OTHER; note = 2'-O-Methyl (2'-OMe) modified RNA base
  modified_base 6: mod_base = OTHER; note = 2'-Flouro (2'-F) modified RNA base
  modified_base 7: mod_base = OTHER; note = 2'-O-Methyl (2'-OMe) modified RNA base
  modified_base 8: mod_base = OTHER; note = 2'-Flouro (2'-F) modified RNA base
  modified_base 9: mod_base = OTHER; note = 2'-O-Methyl (2'-OMe) modified RNA base
  modified_base 10: mod_base = OTHER; note = 2'-Flouro (2'-F) modified RNA base
  modified_base 11: mod_base = OTHER; note = 2'-O-Methyl (2'-OMe) modified RNA base
  modified_base 12: mod_base = OTHER; note = 2'-Flouro (2'-F) modified RNA base
  modified_base 13: mod_base = OTHER; note = 2'-O-Methyl (2'-OMe) modified RNA base
  modified_base 14: mod_base = OTHER; note = 2'-Flouro (2'-F) modified RNA base
  modified_base 15: mod_base = OTHER; note = 2'-O-Methyl (2'-OMe) modified RNA base
  modified_base 16: mod_base = OTHER; note = 2'-Flouro (2'-F) modified RNA base
  modified_base 17: mod_base = OTHER; note = 2'-O-Methyl (2'-OMe) modified RNA base
  modified_base 18: mod_base = OTHER; note = 2'-Flouro (2'-F) modified RNA base
  modified_base 19: mod_base = OTHER; note = 2'-O-Methyl (2'-OMe) modified RNA base
  source 1..21: mol_type = other RNA
attctgaaaccaaatctggtt

SEQ ID NO: 46 - RNA, 21 nt, synthetic construct
  modified_base 20..21: mod_base = OTHER; note = thymine
  modified_base 1: mod_base = OTHER; note = Locked Nucleic Acid (LNA) base
  modified_base 2: mod_base = OTHER; note = 2'-O-Methyl (2'-OMe) modified RNA base
  modified_base 3: mod_base = OTHER; note = 2'-Flouro (2'-F) modified RNA base
  modified_base 4: mod_base = OTHER; note = 2'-O-Methyl (2'-OMe) modified RNA base
  modified_base 5: mod_base = OTHER; note = 2'-Flouro (2'-F) modified RNA base
  modified_base 6: mod_base = OTHER; note = 2'-O-Methyl (2'-OMe) modified RNA base
  modified_base 7: mod_base = OTHER; note = 2'-Flouro (2'-F) modified RNA base
  modified_base 8: mod_base = OTHER; note = 2'-O-Methyl (2'-OMe) modified RNA base
  modified_base 9: mod_base = OTHER; note = 2'-Flouro (2'-F) modified RNA base
  modified_base 10: mod_base = OTHER; note = 2'-O-Methyl (2'-OMe) modified RNA base
  modified_base 11: mod_base = OTHER; note = 2'-Flouro (2'-F) modified RNA base
  modified_base 12: mod_base = OTHER; note = 2'-O-Methyl (2'-OMe) modified RNA base
  modified_base 13: mod_base = OTHER; note = 2'-Flouro (2'-F) modified RNA base
  modified_base 14: mod_base = OTHER; note = 2'-O-Methyl (2'-OMe) modified RNA base
  modified_base 15: mod_base = OTHER; note = 2'-Flouro (2'-F) modified RNA base
  modified_base 16: mod_base = OTHER; note = 2'-O-Methyl (2'-OMe) modified RNA base
  modified_base 17: mod_base = OTHER; note = 2'-Flouro (2'-F) modified RNA base
  modified_base 18: mod_base = OTHER; note = 2'-O-Methyl (2'-OMe) modified RNA base
  modified_base 19: mod_base = OTHER; note = 2'-Flouro (2'-F) modified RNA base
  source 1..21: mol_type = other RNA
cagatttggtttcagaatgtt

SEQ ID NO: 47 - RNA, 21 nt, synthetic construct
  modified_base 20..21: mod_base = OTHER; note = thymine
  modified_base 1: mod_base = OTHER; note = 2'-O-Methyl (2'-OMe) modified RNA base
  modified_base 2: mod_base = OTHER; note = 2'-Flouro (2'-F) modified RNA base
  modified_base 3: mod_base = OTHER; note = 2'-O-Methyl (2'-OMe) modified RNA base
  modified_base 4: mod_base = OTHER; note = 2'-Flouro (2'-F) modified RNA base
  modified_base 5: mod_base = OTHER; note = 2'-O-Methyl (2'-OMe) modified RNA base
  modified_base 6: mod_base = OTHER; note = 2'-Flouro (2'-F) modified RNA base
  modified_base 7: mod_base = OTHER; note = 2'-O-Methyl (2'-OMe) modified RNA base
  modified_base 8: mod_base = OTHER; note = 2'-Flouro (2'-F) modified RNA base
  modified_base 9: mod_base = OTHER; note = 2'-O-Methyl (2'-OMe) modified RNA base
  modified_base 10: mod_base = OTHER; note = 2'-Flouro (2'-F) modified RNA base
  modified_base 11: mod_base = OTHER; note = 2'-O-Methyl (2'-OMe) modified RNA base
  modified_base 12: mod_base = OTHER; note = 2'-Flouro (2'-F) modified RNA base
  modified_base 13: mod_base = OTHER; note = 2'-O-Methyl (2'-OMe) modified RNA base
  modified_base 14: mod_base = OTHER; note = 2'-Flouro (2'-F) modified RNA base
  modified_base 15: mod_base = OTHER; note = 2'-O-Methyl (2'-OMe) modified RNA base
  modified_base 16: mod_base = OTHER; note = 2'-Flouro (2'-F) modified RNA base
  modified_base 17: mod_base = OTHER; note = 2'-O-Methyl (2'-OMe) modified RNA base
  modified_base 18: mod_base = OTHER; note = 2'-Flouro (2'-F) modified RNA base
  modified_base 19: mod_base = OTHER; note = 2'-O-Methyl (2'-OMe) modified RNA base
  source 1..21: mol_type = other RNA
cattctgaaaccaaatctgtt

SEQ ID NO: 48 - RNA, 21 nt, synthetic construct
  modified_base 20..21: mod_base = OTHER; note = thymine
  modified_base 1: mod_base = OTHER; note = Locked Nucleic Acid (LNA) base
  modified_base 2: mod_base = OTHER; note = 2'-O-Methyl (2'-OMe) modified RNA base
  modified_base 3: mod_base = OTHER; note = 2'-Flouro (2'-F) modified RNA base
  modified_base 4: mod_base = OTHER; note = 2'-O-Methyl (2'-OMe) modified RNA base
  modified_base 5: mod_base = OTHER; note = 2'-Flouro (2'-F) modified RNA base
  modified_base 6: mod_base = OTHER; note = 2'-O-Methyl (2'-OMe) modified RNA base
  modified_base 7: mod_base = OTHER; note = 2'-Flouro (2'-F) modified RNA base
  modified_base 8: mod_base = OTHER; note = 2'-O-Methyl (2'-OMe) modified RNA base
  modified_base 9: mod_base = OTHER; note = 2'-Flouro (2'-F) modified RNA base
  modified_base 10: mod_base = OTHER; note = 2'-O-Methyl (2'-OMe) modified RNA base
  modified_base 11: mod_base = OTHER; note = 2'-Flouro (2'-F) modified RNA base
  modified_base 12: mod_base = OTHER; note = 2'-O-Methyl (2'-OMe) modified RNA base
  modified_base 13: mod_base = OTHER; note = 2'-Flouro (2'-F) modified RNA base
  modified_base 14: mod_base = OTHER; note = 2'-O-Methyl (2'-OMe) modified RNA base
  modified_base 15: mod_base = OTHER; note = 2'-Flouro (2'-F) modified RNA base
  modified_base 16: mod_base = OTHER; note = 2'-O-Methyl (2'-OMe) modified RNA base
  modified_base 17: mod_base = OTHER; note = 2'-Flouro (2'-F) modified RNA base
  modified_base 18: mod_base = OTHER; note = 2'-O-Methyl (2'-OMe) modified RNA base
  modified_base 19: mod_base = OTHER; note = 2'-Flouro (2'-F) modified RNA base
  source 1..21: mol_type = other RNA
agatttggtttcagaatgatt

SEQ ID NO: 49 - RNA, 21 nt, synthetic construct
  modified_base 20..21: mod_base = OTHER; note = thymine
  modified_base 1: mod_base = OTHER; note = 2'-O-Methyl (2'-OMe) modified RNA base
  modified_base 2: mod_base = OTHER; note = 2'-Flouro (2'-F) modified RNA base
  modified_base 3: mod_base = OTHER; note = 2'-O-Methyl (2'-OMe) modified RNA base
  modified_base 4: mod_base = OTHER; note = 2'-Flouro (2'-F) modified RNA base
  modified_base 5: mod_base = OTHER; note = 2'-O-Methyl (2'-OMe) modified RNA base
  modified_base 6: mod_base = OTHER; note = 2'-Flouro (2'-F) modified RNA base
  modified_base 7: mod_base = OTHER; note = 2'-O-Methyl (2'-OMe) modified RNA base
  modified_base 8: mod_base = OTHER; note = 2'-Flouro (2'-F) modified RNA base
  modified_base 9: mod_base = OTHER; note = 2'-O-Methyl (2'-OMe) modified RNA base
  modified_base 10: mod_base = OTHER; note = 2'-Flouro (2'-F) modified RNA base
  modified_base 11: mod_base = OTHER; note = 2'-O-Methyl (2'-OMe) modified RNA base
  modified_base 12: mod_base = OTHER; note = 2'-Flouro (2'-F) modified RNA base
  modified_base 13: mod_base = OTHER; note = 2'-O-Methyl (2'-OMe) modified RNA base
  modified_base 14: mod_base = OTHER; note = 2'-Flouro (2'-F) modified RNA base
  modified_base 15: mod_base = OTHER; note = 2'-O-Methyl (2'-OMe) modified RNA base
  modified_base 16: mod_base = OTHER; note = 2'-Flouro (2'-F) modified RNA base
  modified_base 17: mod_base = OTHER; note = 2'-O-Methyl (2'-OMe) modified RNA base
  modified_base 18: mod_base = OTHER; note = 2'-Flouro (2'-F) modified RNA base
  modified_base 19: mod_base = OTHER; note = 2'-O-Methyl (2'-OMe) modified RNA base
  source 1..21: mol_type = other RNA
tcattctgaaaccaaatcttt

SEQ ID NO: 50 - RNA, 21 nt, synthetic construct
  modified_base 20..21: mod_base = OTHER; note = thymine
  modified_base 1: mod_base = OTHER; note = Locked Nucleic Acid (LNA) base
  modified_base 2: mod_base = OTHER; note = 2'-O-Methyl (2'-OMe) modified RNA base
  modified_base 3: mod_base = OTHER; note = 2'-Flouro (2'-F) modified RNA base
  modified_base 4: mod_base = OTHER; note = 2'-O-Methyl (2'-OMe) modified RNA base
  modified_base 5: mod_base = OTHER; note = 2'-Flouro (2'-F) modified RNA base
  modified_base 6: mod_base = OTHER; note = 2'-O-Methyl (2'-OMe) modified RNA base
  modified_base 7: mod_base = OTHER; note = 2'-Flouro (2'-F) modified RNA base
  modified_base 8: mod_base = OTHER; note = 2'-O-Methyl (2'-OMe) modified RNA base
  modified_base 9: mod_base = OTHER; note = 2'-Flouro (2'-F) modified RNA base
  modified_base 10: mod_base = OTHER; note = 2'-O-Methyl (2'-OMe) modified RNA base
  modified_base 11: mod_base = OTHER; note = 2'-Flouro (2'-F) modified RNA base
  modified_base 12: mod_base = OTHER; note = 2'-O-Methyl (2'-OMe) modified RNA base
  modified_base 13: mod_base = OTHER; note = 2'-Flouro (2'-F) modified RNA base
  modified_base 14: mod_base = OTHER; note = 2'-O-Methyl (2'-OMe) modified RNA base
  modified_base 15: mod_base = OTHER; note = 2'-Flouro (2'-F) modified RNA base
  modified_base 16: mod_base = OTHER; note = 2'-O-Methyl (2'-OMe) modified RNA base
  modified_base 17: mod_base = OTHER; note = 2'-Flouro (2'-F) modified RNA base
  modified_base 18: mod_base = OTHER; note = 2'-O-Methyl (2'-OMe) modified RNA base
  modified_base 19: mod_base = OTHER; note = 2'-Flouro (2'-F) modified RNA base
  source 1..21: mol_type = other RNA
gatttggtttcagaatgagtt

SEQ ID NO: 51 - RNA, 21 nt, synthetic construct
  modified_base 20..21: mod_base = OTHER; note = thymine
  modified_base 1: mod_base = OTHER; note = 2'-O-Methyl (2'-OMe) modified RNA base
  modified_base 2: mod_base = OTHER; note = 2'-Flouro (2'-F) modified RNA base
  modified_base 3: mod_base = OTHER; note = 2'-O-Methyl (2'-OMe) modified RNA base
  modified_base 4: mod_base = OTHER; note = 2'-Flouro (2'-F) modified RNA base
  modified_base 5: mod_base = OTHER; note = 2'-O-Methyl (2'-OMe) modified RNA base
  modified_base 6: mod_base = OTHER; note = 2'-Flouro (2'-F) modified RNA base
  modified_base 7: mod_base = OTHER; note = 2'-O-Methyl (2'-OMe) modified RNA base
  modified_base 8: mod_base = OTHER; note = 2'-Flouro (2'-F) modified RNA base
  modified_base 9: mod_base = OTHER; note = 2'-O-Methyl (2'-OMe) modified RNA base
  modified_base 10: mod_base = OTHER; note = 2'-Flouro (2'-F) modified RNA base
  modified_base 11: mod_base = OTHER; note = 2'-O-Methyl (2'-OMe) modified RNA base
  modified_base 12: mod_base = OTHER; note = 2'-Flouro (2'-F) modified RNA base
  modified_base 13: mod_base = OTHER; note = 2'-O-Methyl (2'-OMe) modified RNA base
  modified_base 14: mod_base = OTHER; note = 2'-Flouro (2'-F) modified RNA base
  modified_base 15: mod_base = OTHER; note = 2'-O-Methyl (2'-OMe) modified RNA base
  modified_base 16: mod_base = OTHER; note = 2'-Flouro (2'-F) modified RNA base
  modified_base 17: mod_base = OTHER; note = 2'-O-Methyl (2'-OMe) modified RNA base
  modified_base 18: mod_base = OTHER; note = 2'-Flouro (2'-F) modified RNA base
  modified_base 19: mod_base = OTHER; note = 2'-O-Methyl (2'-OMe) modified RNA base
  source 1..21: mol_type = other RNA
ctcattctgaaaccaaatctt

SEQ ID NO: 52 - RNA, 21 nt, synthetic construct
  modified_base 20..21: mod_base = OTHER; note = thymine
  modified_base 1: mod_base = OTHER; note = Locked Nucleic Acid (LNA) base
  modified_base 2: mod_base = OTHER; note = 2'-O-Methyl (2'-OMe) modified RNA base
  modified_base 3: mod_base = OTHER; note = 2'-Flouro (2'-F) modified RNA base
  modified_base 4: mod_base = OTHER; note = 2'-O-Methyl (2'-OMe) modified RNA base
  modified_base 5: mod_base = OTHER; note = 2'-Flouro (2'-F) modified RNA base
  modified_base 6: mod_base = OTHER; note = 2'-O-Methyl (2'-OMe) modified RNA base
  modified_base 7: mod_base = OTHER; note = 2'-Flouro (2'-F) modified RNA base
  modified_base 8: mod_base = OTHER; note = 2'-O-Methyl (2'-OMe) modified RNA base
  modified_base 9: mod_base = OTHER; note = 2'-Flouro (2'-F) modified RNA base
  modified_base 10: mod_base = OTHER; note = 2'-O-Methyl (2'-OMe) modified RNA base
  modified_base 11: mod_base = OTHER; note = 2'-Flouro (2'-F) modified RNA base
  modified_base 12: mod_base = OTHER; note = 2'-O-Methyl (2'-OMe) modified RNA base
  modified_base 13: mod_base = OTHER; note = 2'-Flouro (2'-F) modified RNA base
  modified_base 14: mod_base = OTHER; note = 2'-O-Methyl (2'-OMe) modified RNA base
  modified_base 15: mod_base = OTHER; note = 2'-Flouro (2'-F) modified RNA base
  modified_base 16: mod_base = OTHER; note = 2'-O-Methyl (2'-OMe) modified RNA base
  modified_base 17: mod_base = OTHER; note = 2'-Flouro (2'-F) modified RNA base
  modified_base 18: mod_base = OTHER; note = 2'-O-Methyl (2'-OMe) modified RNA base
  modified_base 19: mod_base = OTHER; note = 2'-Flouro (2'-F) modified RNA base
  source 1..21: mol_type = other RNA
ggattcagatctggtttcatt

SEQ ID NO: 53 - RNA, 21 nt, synthetic construct
  modified_base 20..21: mod_base = OTHER; note = thymine
  modified_base 1: mod_base = OTHER; note = 2'-O-Methyl (2'-OMe) modified RNA base
  modified_base 2: mod_base = OTHER; note = 2'-Flouro (2'-F) modified RNA base
  modified_base 3: mod_base = OTHER; note = 2'-O-Methyl (2'-OMe) modified RNA base
  modified_base 4: mod_base = OTHER; note = 2'-Flouro (2'-F) modified RNA base
  modified_base 5: mod_base = OTHER; note = 2'-O-Methyl (2'-OMe) modified RNA base
  modified_base 6: mod_base = OTHER; note = 2'-Flouro (2'-F) modified RNA base
  modified_base 7: mod_base = OTHER; note = 2'-O-Methyl (2'-OMe) modified RNA base
  modified_base 8: mod_base = OTHER; note = 2'-Flouro (2'-F) modified RNA base
  modified_base 9: mod_base = OTHER; note = 2'-O-Methyl (2'-OMe) modified RNA base
  modified_base 10: mod_base = OTHER; note = 2'-Flouro (2'-F) modified RNA base
  modified_base 11: mod_base = OTHER; note = 2'-O-Methyl (2'-OMe) modified RNA base
  modified_base 12: mod_base = OTHER; note = 2'-Flouro (2'-F) modified RNA base
  modified_base 13: mod_base = OTHER; note = 2'-O-Methyl (2'-OMe) modified RNA base
  modified_base 14: mod_base = OTHER; note = 2'-Flouro (2'-F) modified RNA base
  modified_base 15: mod_base = OTHER; note = 2'-O-Methyl (2'-OMe) modified RNA base
  modified_base 16: mod_base = OTHER; note = 2'-Flouro (2'-F) modified RNA base
  modified_base 17: mod_base = OTHER; note = 2'-O-Methyl (2'-OMe) modified RNA base
  modified_base 18: mod_base = OTHER; note = 2'-Flouro (2'-F) modified RNA base
  modified_base 19: mod_base = OTHER; note = 2'-O-Methyl (2'-OMe) modified RNA base
  source 1..21: mol_type = other RNA
tgaaaccagatctgaatcctt

SEQ ID NO: 54 - RNA, 21 nt, synthetic construct
  modified_base 20..21: mod_base = OTHER; note = thymine
  modified_base 1: mod_base = OTHER; note = Locked Nucleic Acid (LNA) base
  modified_base 2: mod_base = OTHER; note = 2'-O-Methyl (2'-OMe) modified RNA base
  modified_base 3: mod_base = OTHER; note = 2'-Flouro (2'-F) modified RNA base
  modified_base 4: mod_base = OTHER; note = 2'-O-Methyl (2'-OMe) modified RNA base
  modified_base 5: mod_base = OTHER; note = 2'-Flouro (2'-F) modified RNA base
  modified_base 6: mod_base = OTHER; note = 2'-O-Methyl (2'-OMe) modified RNA base
  modified_base 7: mod_base = OTHER; note = 2'-Flouro (2'-F) modified RNA base
  modified_base 8: mod_base = OTHER; note = 2'-O-Methyl (2'-OMe) modified RNA base
  modified_base 9: mod_base = OTHER; note = 2'-Flouro (2'-F) modified RNA base
  modified_base 10: mod_base = OTHER; note = 2'-O-Methyl (2'-OMe) modified RNA base
  modified_base 11: mod_base = OTHER; note = 2'-Flouro (2'-F) modified RNA base
  modified_base 12: mod_base = OTHER; note = 2'-O-Methyl (2'-OMe) modified RNA base
  modified_base 13: mod_base = OTHER; note = 2'-Flouro (2'-F) modified RNA base
  modified_base 14: mod_base = OTHER; note = 2'-O-Methyl (2'-OMe) modified RNA base
  modified_base 15: mod_base = OTHER; note = 2'-Flouro (2'-F) modified RNA base
  modified_base 16: mod_base = OTHER; note = 2'-O-Methyl (2'-OMe) modified RNA base
  modified_base 17: mod_base = OTHER; note = 2'-Flouro (2'-F) modified RNA base
  modified_base 18: mod_base = OTHER; note = 2'-O-Methyl (2'-OMe) modified RNA base
  modified_base 19: mod_base = OTHER; note = 2'-Flouro (2'-F) modified RNA base
  source 1..21: mol_type = other RNA
gatctggtttcagaatcgatt

SEQ ID NO: 55 - RNA, 21 nt, synthetic construct
  modified_base 20..21: mod_base = OTHER; note = thymine
  modified_base 1: mod_base = OTHER; note = 2'-O-Methyl (2'-OMe) modified RNA base
  modified_base 2: mod_base = OTHER; note = 2'-Flouro (2'-F) modified RNA base
  modified_base 3: mod_base = OTHER; note = 2'-O-Methyl (2'-OMe) modified RNA base
  modified_base 4: mod_base = OTHER; note = 2'-Flouro (2'-F) modified RNA base
  modified_base 5: mod_base = OTHER; note = 2'-O-Methyl (2'-OMe) modified RNA base
  modified_base 6: mod_base = OTHER; note = 2'-Flouro (2'-F) modified RNA base
  modified_base 7: mod_base = OTHER; note = 2'-O-Methyl (2'-OMe) modified RNA base
  modified_base 8: mod_base = OTHER; note = 2'-Flouro (2'-F) modified RNA base
  modified_base 9: mod_base = OTHER; note = 2'-O-Methyl (2'-OMe) modified RNA base
  modified_base 10: mod_base = OTHER; note = 2'-Flouro (2'-F) modified RNA base
  modified_base 11: mod_base = OTHER; note = 2'-O-Methyl (2'-OMe) modified RNA base
  modified_base 12: mod_base = OTHER; note = 2'-Flouro (2'-F) modified RNA base
  modified_base 13: mod_base = OTHER; note = 2'-O-Methyl (2'-OMe) modified RNA base
  modified_base 14: mod_base = OTHER; note = 2'-Flouro (2'-F) modified RNA base
  modified_base 15: mod_base = OTHER; note = 2'-O-Methyl (2'-OMe) modified RNA base
  modified_base 16: mod_base = OTHER; note = 2'-Flouro (2'-F) modified RNA base
  modified_base 17: mod_base = OTHER; note = 2'-O-Methyl (2'-OMe) modified RNA base
  modified_base 18: mod_base = OTHER; note = 2'-Flouro (2'-F) modified RNA base
  modified_base 19: mod_base = OTHER; note = 2'-O-Methyl (2'-OMe) modified RNA base
  source 1..21: mol_type = other RNA
tcgattctgaaaccagatctt

SEQ ID NO: 56 - RNA, 21 nt, synthetic construct
  modified_base 20..21: mod_base = OTHER; note = thymine
  modified_base 1: mod_base = OTHER; note = Locked Nucleic Acid (LNA) base
  modified_base 2: mod_base = OTHER; note = 2'-O-Methyl (2'-OMe) modified RNA base
  modified_base 3: mod_base = OTHER; note = 2'-Flouro (2'-F) modified RNA base
  modified_base 4: mod_base = OTHER; note = 2'-O-Methyl (2'-OMe) modified RNA base
  modified_base 5: mod_base = OTHER; note = 2'-Flouro (2'-F) modified RNA base
  modified_base 6: mod_base = OTHER; note = 2'-O-Methyl (2'-OMe) modified RNA base
  modified_base 7: mod_base = OTHER; note = 2'-Flouro (2'-F) modified RNA base
  modified_base 8: mod_base = OTHER; note = 2'-O-Methyl (2'-OMe) modified RNA base
  modified_base 9: mod_base = OTHER; note = 2'-Flouro (2'-F) modified RNA base
  modified_base 10: mod_base = OTHER; note = 2'-O-Methyl (2'-OMe) modified RNA base
  modified_base 11: mod_base = OTHER; note = 2'-Flouro (2'-F) modified RNA base
  modified_base 12: mod_base = OTHER; note = 2'-O-Methyl (2'-OMe) modified RNA base
  modified_base 13: mod_base = OTHER; note = 2'-Flouro (2'-F) modified RNA base
  modified_base 14: mod_base = OTHER; note = 2'-O-Methyl (2'-OMe) modified RNA base
  modified_base 15: mod_base = OTHER; note = 2'-Flouro (2'-F) modified RNA base
  modified_base 16: mod_base = OTHER; note = 2'-O-Methyl (2'-OMe) modified RNA base
  modified_base 17: mod_base = OTHER; note = 2'-Flouro (2'-F) modified RNA base
  modified_base 18: mod_base = OTHER; note = 2'-O-Methyl (2'-OMe) modified RNA base
  modified_base 19: mod_base = OTHER; note = 2'-Flouro (2'-F) modified RNA base
  source 1..21: mol_type = other RNA
atctggtttcagaatcgaatt

SEQ ID NO: 57 - RNA, 21 nt, synthetic construct
  modified_base 20..21: mod_base = OTHER; note = thymine
  modified_base 1: mod_base = OTHER; note = 2'-O-Methyl (2'-OMe) modified RNA base
  modified_base 2: mod_base = OTHER; note = 2'-Flouro (2'-F) modified RNA base
  modified_base 3: mod_base = OTHER; note = 2'-O-Methyl (2'-OMe) modified RNA base
  modified_base 4: mod_base = OTHER; note = 2'-Flouro (2'-F) modified RNA base
  modified_base 5: mod_base = OTHER; note = 2'-O-Methyl (2'-OMe) modified RNA base
  modified_base 6: mod_base = OTHER; note = 2'-Flouro (2'-F) modified RNA base
  modified_base 7: mod_base = OTHER; note = 2'-O-Methyl (2'-OMe) modified RNA base
  modified_base 8: mod_base = OTHER; note = 2'-Flouro (2'-F) modified RNA base
  modified_base 9: mod_base = OTHER; note = 2'-O-Methyl (2'-OMe) modified RNA base
  modified_base 10: mod_base = OTHER; note = 2'-Flouro (2'-F) modified RNA base
  modified_base 11: mod_base = OTHER; note = 2'-O-Methyl (2'-OMe) modified RNA base
  modified_base 12: mod_base = OTHER; note = 2'-Flouro (2'-F) modified RNA base
  modified_base 13: mod_base = OTHER; note = 2'-O-Methyl (2'-OMe) modified RNA base
  modified_base 14: mod_base = OTHER; note = 2'-Flouro (2'-F) modified RNA base
  modified_base 15: mod_base = OTHER; note = 2'-O-Methyl (2'-OMe) modified RNA base
  modified_base 16: mod_base = OTHER; note = 2'-Flouro (2'-F) modified RNA base
  modified_base 17: mod_base = OTHER; note = 2'-O-Methyl (2'-OMe) modified RNA base
  modified_base 18: mod_base = OTHER; note = 2'-Flouro (2'-F) modified RNA base
  modified_base 19: mod_base = OTHER; note = 2'-O-Methyl (2'-OMe) modified RNA base
  source 1..21: mol_type = other RNA
ttcgattctgaaaccagattt

SEQ ID NO: 58 - RNA, 21 nt, synthetic construct
  modified_base 20..21: mod_base = OTHER; note = thymine
  modified_base 1: mod_base = OTHER; note = Locked Nucleic Acid (LNA) base
  modified_base 2: mod_base = OTHER; note = 2'-O-Methyl (2'-OMe) modified RNA base
  modified_base 3: mod_base = OTHER; note = 2'-Flouro (2'-F) modified RNA base
  modified_base 4: mod_base = OTHER; note = 2'-O-Methyl (2'-OMe) modified RNA base
  modified_base 5: mod_base = OTHER; note = 2'-Flouro (2'-F) modified RNA base
  modified_base 6: mod_base = OTHER; note = 2'-O-Methyl (2'-OMe) modified RNA base
  modified_base 7: mod_base = OTHER; note = 2'-Flouro (2'-F) modified RNA base
  modified_base 8: mod_base = OTHER; note = 2'-O-Methyl (2'-OMe) modified RNA base
  modified_base 9: mod_base = OTHER; note = 2'-Flouro (2'-F) modified RNA base
  modified_base 10: mod_base = OTHER; note = 2'-O-Methyl (2'-OMe) modified RNA base
  modified_base 11: mod_base = OTHER; note = 2'-Flouro (2'-F) modified RNA base
  modified_base 12: mod_base = OTHER; note = 2'-O-Methyl (2'-OMe) modified RNA base
  modified_base 13: mod_base = OTHER; note = 2'-Flouro (2'-F) modified RNA base
  modified_base 14: mod_base = OTHER; note = 2'-O-Methyl (2'-OMe) modified RNA base
  modified_base 15: mod_base = OTHER; note = 2'-Flouro (2'-F) modified RNA base
  modified_base 16: mod_base = OTHER; note = 2'-O-Methyl (2'-OMe) modified RNA base
  modified_base 17: mod_base = OTHER; note = 2'-Flouro (2'-F) modified RNA base
  modified_base 18: mod_base = OTHER; note = 2'-O-Methyl (2'-OMe) modified RNA base
  modified_base 19: mod_base = OTHER; note = 2'-Flouro (2'-F) modified RNA base
  source 1..21: mol_type = other RNA
aggcgcaacctctcctagatt

SEQ ID NO: 59 - RNA, 21 nt, synthetic construct
  modified_base 20..21: mod_base = OTHER; note = thymine
  modified_base 1: mod_base = OTHER; note = 2'-O-Methyl (2'-OMe) modified RNA base
  modified_base 2: mod_base = OTHER; note = 2'-Flouro (2'-F) modified RNA base
  modified_base 3: mod_base = OTHER; note = 2'-O-Methyl (2'-OMe) modified RNA base
  modified_base 4: mod_base = OTHER; note = 2'-Flouro (2'-F) modified RNA base
  modified_base 5: mod_base = OTHER; note = 2'-O-Methyl (2'-OMe) modified RNA base
  modified_base 6: mod_base = OTHER; note = 2'-Flouro (2'-F) modified RNA base
  modified_base 7: mod_base = OTHER; note = 2'-O-Methyl (2'-OMe) modified RNA base
  modified_base 8: mod_base = OTHER; note = 2'-Flouro (2'-F) modified RNA base
  modified_base 9: mod_base = OTHER; note = 2'-O-Methyl (2'-OMe) modified RNA base
  modified_base 10: mod_base = OTHER; note = 2'-Flouro (2'-F) modified RNA base
  modified_base 11: mod_base = OTHER; note = 2'-O-Methyl (2'-OMe) modified RNA base
  modified_base 12: mod_base = OTHER; note = 2'-Flouro (2'-F) modified RNA base
  modified_base 13: mod_base = OTHER; note = 2'-O-Methyl (2'-OMe) modified RNA base
  modified_base 14: mod_base = OTHER; note = 2'-Flouro (2'-F) modified RNA base
  modified_base 15: mod_base = OTHER; note = 2'-O-Methyl (2'-OMe) modified RNA base
  modified_base 16: mod_base = OTHER; note = 2'-Flouro (2'-F) modified RNA base
  modified_base 17: mod_base = OTHER; note = 2'-O-Methyl (2'-OMe) modified RNA base
  modified_base 18: mod_base = OTHER; note = 2'-Flouro (2'-F) modified RNA base
  modified_base 19: mod_base = OTHER; note = 2'-O-Methyl (2'-OMe) modified RNA base
  source 1..21: mol_type = other RNA
tctaggagaggttgcgccttt

SEQ ID NO: 60 - RNA, 21 nt, synthetic construct
  modified_base 20..21: mod_base = OTHER; note = thymine
  modified_base 1: mod_base = OTHER; note = Locked Nucleic Acid (LNA) base
  modified_base 2: mod_base = OTHER; note = 2'-O-Methyl (2'-OMe) modified RNA base
  modified_base 3: mod_base = OTHER; note = 2'-Flouro (2'-F) modified RNA base
  modified_base 4: mod_base = OTHER; note = 2'-O-Methyl (2'-OMe) modified RNA base
  modified_base 5: mod_base = OTHER; note = 2'-Flouro (2'-F) modified RNA base
  modified_base 6: mod_base = OTHER; note = 2'-O-Methyl (2'-OMe) modified RNA base
  modified_base 7: mod_base = OTHER; note = 2'-Flouro (2'-F) modified RNA base
  modified_base 8: mod_base = OTHER; note = 2'-O-Methyl (2'-OMe) modified RNA base
  modified_base 9: mod_base = OTHER; note = 2'-Flouro (2'-F) modified RNA base
  modified_base 10: mod_base = OTHER; note = 2'-O-Methyl (2'-OMe) modified RNA base
  modified_base 11: mod_base = OTHER; note = 2'-Flouro (2'-F) modified RNA base
  modified_base 12: mod_base = OTHER; note = 2'-O-Methyl (2'-OMe) modified RNA base
  modified_base 13: mod_base = OTHER; note = 2'-Flouro (2'-F) modified RNA base
  modified_base 14: mod_base = OTHER; note = 2'-O-Methyl (2'-OMe) modified RNA base
  modified_base 15: mod_base = OTHER; note = 2'-Flouro (2'-F) modified RNA base
  modified_base 16: mod_base = OTHER; note = 2'-O-Methyl (2'-OMe) modified RNA base
  modified_base 17: mod_base = OTHER; note = 2'-Flouro (2'-F) modified RNA base
  modified_base 18: mod_base = OTHER; note = 2'-O-Methyl (2'-OMe) modified RNA base
  modified_base 19: mod_base = OTHER; note = 2'-Flouro (2'-F) modified RNA base
  source 1..21: mol_type = other RNA
cgaggaagaataccgggcttt

SEQ ID NO: 61 - RNA, 21 nt, synthetic construct
  modified_base 20..21: mod_base = OTHER; note = thymine
  modified_base 1: mod_base = OTHER; note = 2'-O-Methyl (2'-OMe) modified RNA base
  modified_base 2: mod_base = OTHER; note = 2'-Flouro (2'-F) modified RNA base
  modified_base 3: mod_base = OTHER; note = 2'-O-Methyl (2'-OMe) modified RNA base
  modified_base 4: mod_base = OTHER; note = 2'-Flouro (2'-F) modified RNA base
  modified_base 5: mod_base = OTHER; note = 2'-O-Methyl (2'-OMe) modified RNA base
  modified_base 6: mod_base = OTHER; note = 2'-Flouro (2'-F) modified RNA base
  modified_base 7: mod_base = OTHER; note = 2'-O-Methyl (2'-OMe) modified RNA base
  modified_base 8: mod_base = OTHER; note = 2'-Flouro (2'-F) modified RNA base
  modified_base 9: mod_base = OTHER; note = 2'-O-Methyl (2'-OMe) modified RNA base
  modified_base 10: mod_base = OTHER; note = 2'-Flouro (2'-F) modified RNA base
  modified_base 11: mod_base = OTHER; note = 2'-O-Methyl (2'-OMe) modified RNA base
  modified_base 12: mod_base = OTHER; note = 2'-Flouro (2'-F) modified RNA base
  modified_base 13: mod_base = OTHER; note = 2'-O-Methyl (2'-OMe) modified RNA base
  modified_base 14: mod_base = OTHER; note = 2'-Flouro (2'-F) modified RNA base
  modified_base 15: mod_base = OTHER; note = 2'-O-Methyl (2'-OMe) modified RNA base
  modified_base 16: mod_base = OTHER; note = 2'-Flouro (2'-F) modified RNA base
  modified_base 17: mod_base = OTHER; note = 2'-O-Methyl (2'-OMe) modified RNA base
  modified_base 18: mod_base = OTHER; note = 2'-Flouro (2'-F) modified RNA base
  modified_base 19: mod_base = OTHER; note = 2'-O-Methyl (2'-OMe) modified RNA base
  source 1..21: mol_type = other RNA
agcccggtattcttcctcgtt

SEQ ID NO: 62 - RNA, 21 nt, synthetic construct
  modified_base 20..21: mod_base = OTHER; note = thymine
  modified_base 1: mod_base = OTHER; note = Locked Nucleic Acid (LNA) base
  modified_base 2: mod_base = OTHER; note = 2'-O-Methyl (2'-OMe) modified RNA base
  modified_base 3: mod_base = OTHER; note = 2'-Flouro (2'-F) modified RNA base
  modified_base 4: mod_base = OTHER; note = 2'-O-Methyl (2'-OMe) modified RNA base
  modified_base 5: mod_base = OTHER; note = 2'-Flouro (2'-F) modified RNA base
  modified_base 6: mod_base = OTHER; note = 2'-O-Methyl (2'-OMe) modified RNA base
  modified_base 7: mod_base = OTHER; note = 2'-Flouro (2'-F) modified RNA base
  modified_base 8: mod_base = OTHER; note = 2'-O-Methyl (2'-OMe) modified RNA base
  modified_base 9: mod_base = OTHER; note = 2'-Flouro (2'-F) modified RNA base
  modified_base 10: mod_base = OTHER; note = 2'-O-Methyl (2'-OMe) modified RNA base
  modified_base 11: mod_base = OTHER; note = 2'-Flouro (2'-F) modified RNA base
  modified_base 12: mod_base = OTHER; note = 2'-O-Methyl (2'-OMe) modified RNA base
  modified_base 13: mod_base = OTHER; note = 2'-Flouro (2'-F) modified RNA base
  modified_base 14: mod_base = OTHER; note = 2'-O-Methyl (2'-OMe) modified RNA base
  modified_base 15: mod_base = OTHER; note = 2'-Flouro (2'-F) modified RNA base
  modified_base 16: mod_base = OTHER; note = 2'-O-Methyl (2'-OMe) modified RNA base
  modified_base 17: mod_base = OTHER; note = 2'-Flouro (2'-F) modified RNA base
  modified_base 18: mod_base = OTHER; note = 2'-O-Methyl (2'-OMe) modified RNA base
  modified_base 19: mod_base = OTHER; note = 2'-Flouro (2'-F) modified RNA base
  source 1..21: mol_type = other RNA
ctctgctggaggagctttatt

SEQ ID NO: 63 - RNA, 21 nt, synthetic construct
  modified_base 20..21: mod_base = OTHER; note = thymine
  modified_base 1: mod_base = OTHER; note = 2'-O-Methyl (2'-OMe) modified RNA base
  modified_base 2: mod_base = OTHER; note = 2'-Flouro (2'-F) modified RNA base
  modified_base 3: mod_base = OTHER; note = 2'-O-Methyl (2'-OMe) modified RNA base
  modified_base 4: mod_base = OTHER; note = 2'-Flouro (2'-F) modified RNA base
  modified_base 5: mod_base = OTHER; note = 2'-O-Methyl (2'-OMe) modified RNA base
  modified_base 6: mod_base = OTHER; note = 2'-Flouro (2'-F) modified RNA base
  modified_base 7: mod_base = OTHER; note = 2'-O-Methyl (2'-OMe) modified RNA base
  modified_base 8: mod_base = OTHER; note = 2'-Flouro (2'-F) modified RNA base
  modified_base 9: mod_base = OTHER; note = 2'-O-Methyl (2'-OMe) modified RNA base
  modified_base 10: mod_base = OTHER; note = 2'-Flouro (2'-F) modified RNA base
  modified_base 11: mod_base = OTHER; note = 2'-O-Methyl (2'-OMe) modified RNA base
  modified_base 12: mod_base = OTHER; note = 2'-Flouro (2'-F) modified RNA base
  modified_base 13: mod_base = OTHER; note = 2'-O-Methyl (2'-OMe) modified RNA base
  modified_base 14: mod_base = OTHER; note = 2'-Flouro (2'-F) modified RNA base
  modified_base 15: mod_base = OTHER; note = 2'-O-Methyl (2'-OMe) modified RNA base
  modified_base 16: mod_base = OTHER; note = 2'-Flouro (2'-F) modified RNA base
  modified_base 17: mod_base = OTHER; note = 2'-O-Methyl (2'-OMe) modified RNA base
  modified_base 18: mod_base = OTHER; note = 2'-Flouro (2'-F) modified RNA base
  modified_base 19: mod_base = OTHER; note = 2'-O-Methyl (2'-OMe) modified RNA base
  source 1..21: mol_type = other RNA
taaagctcctccagcagagtt

SEQ ID NO: 64 - RNA, 21 nt, synthetic construct
  modified_base 20..21: mod_base = OTHER; note = thymine
  modified_base 1: mod_base = OTHER; note = Locked Nucleic Acid (LNA) base
  modified_base 2: mod_base = OTHER; note = 2'-O-Methyl (2'-OMe) modified RNA base
  modified_base 3: mod_base = OTHER; note = 2'-Flouro (2'-F) modified RNA base
  modified_base 4: mod_base = OTHER; note = 2'-O-Methyl (2'-OMe) modified RNA base
  modified_base 5: mod_base = OTHER; note = 2'-Flouro (2'-F) modified RNA base
  modified_base 6: mod_base = OTHER; note = 2'-O-Methyl (2'-OMe) modified RNA base
  modified_base 7: mod_base = OTHER; note = 2'-Flouro (2'-F) modified RNA base
  modified_base 8: mod_base = OTHER; note = 2'-O-Methyl (2'-OMe) modified RNA base
  modified_base 9: mod_base = OTHER; note = 2'-Flouro (2'-F) modified RNA base
  modified_base 10: mod_base = OTHER; note = 2'-O-Methyl (2'-OMe) modified RNA base
  modified_base 11: mod_base = OTHER; note = 2'-Flouro (2'-F) modified RNA base
  modified_base 12: mod_base = OTHER; note = 2'-O-Methyl (2'-OMe) modified RNA base
  modified_base 13: mod_base = OTHER; note = 2'-Flouro (2'-F) modified RNA base
  modified_base 14: mod_base = OTHER; note = 2'-O-Methyl (2'-OMe) modified RNA base
  modified_base 15: mod_base = OTHER; note = 2'-Flouro (2'-F) modified RNA base
  modified_base 16: mod_base = OTHER; note = 2'-O-Methyl (2'-OMe) modified RNA base
  modified_base 17: mod_base = OTHER; note = 2'-Flouro (2'-F) modified RNA base
  modified_base 18: mod_base = OTHER; note = 2'-O-Methyl (2'-OMe) modified RNA base
  modified_base 19: mod_base = OTHER; note = 2'-Flouro (2'-F) modified RNA base
  source 1..21: mol_type = other RNA
cgcggtgttctgcctttcttt

SEQ ID NO: 65 - RNA, 21 nt, synthetic construct
  modified_base 20..21: mod_base = OTHER; note = thymine
  modified_base 1: mod_base = OTHER; note = 2'-O-Methyl (2'-OMe) modified RNA base
  modified_base 2: mod_base = OTHER; note = 2'-Flouro (2'-F) modified RNA base
  modified_base 3: mod_base = OTHER; note = 2'-O-Methyl (2'-OMe) modified RNA base
  modified_base 4: mod_base = OTHER; note = 2'-Flouro (2'-F) modified RNA base
  modified_base 5: mod_base = OTHER; note = 2'-O-Methyl (2'-OMe) modified RNA base
  modified_base 6: mod_base = OTHER; note = 2'-Flouro (2'-F) modified RNA base
  modified_base 7: mod_base = OTHER; note = 2'-O-Methyl (2'-OMe) modified RNA base
  modified_base 8: mod_base = OTHER; note = 2'-Flouro (2'-F) modified RNA base
  modified_base 9: mod_base = OTHER; note = 2'-O-Methyl (2'-OMe) modified RNA base
  modified_base 10: mod_base = OTHER; note = 2'-Flouro (2'-F) modified RNA base
  modified_base 11: mod_base = OTHER; note = 2'-O-Methyl (2'-OMe) modified RNA base
  modified_base 12: mod_base = OTHER; note = 2'-Flouro (2'-F) modified RNA base
  modified_base 13: mod_base = OTHER; note = 2'-O-Methyl (2'-OMe) modified RNA base
  modified_base 14: mod_base = OTHER; note = 2'-Flouro (2'-F) modified RNA base
  modified_base 15: mod_base = OTHER; note = 2'-O-Methyl (2'-OMe) modified RNA base
  modified_base 16: mod_base = OTHER; note = 2'-Flouro (2'-F) modified RNA base
  modified_base 17: mod_base = OTHER; note = 2'-O-Methyl (2'-OMe) modified RNA base
  modified_base 18: mod_base = OTHER; note = 2'-Flouro (2'-F) modified RNA base
  modified_base 19: mod_base = OTHER; note = 2'-O-Methyl (2'-OMe) modified RNA base
  source 1..21: mol_type = other RNA
agaatggcagttctccgcgtt

SEQ ID NO: 66 - DNA, 8671 bp, Homo sapiens
  source 1..8671: mol_type = genomic DNA
gggatgcgcgcgcctggggctctcccacagggggctttcgtgagccaggcagcgagggccgcccccgcgctgcagcccagccaggccgcgacggcagagggggtctcccaacctgccccggcgcgcggggatttcgcctacgccgccccggctcctccggacggggcgctctcccaccctcaggctcctcggtggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggttgggacggggtcgggtggttcggggcagggcggtggcctctctttcgcggggaacgcctggctggctacggaggggcgtgtctccgccccgccccctccaccgggctgaccggcctgggattcctgccttctaggtctaggcccggtgagagactccacaccgcggagaactgccattctttcctgggcatcccggggatcccagagccggcccaggtaccagcaggtgggccgcctactgcgcacgcgcgggtttgcgggcagccgcctgggctgtgggagcagcccgggcagagctctcctgcctctccaccagcccaccccgccgcctgaccgccccctccccacccccaccccccacccccggaaaacgcgtcgtcccctgggctgggtggagacccccgtcccgcgaaacaccgggccccgcgcagcgtccgggcctgacaccgctccggcggctcgcctcctctgcgcccccgcgccaccgtcgcccgcccgcccgggcccctgcagccgcccaggtgccagcacggagcgcctggcggcggaacgcagaccccaggcccggcgcacaccggggacgctgagcgttccaggcgggagggaaggcgggcagagatggagagaggaacgggagacctagaggggcggaaggacgggcggagggacgttaggagggagggagggaggcagggaggcagggaggaacggagggaaagacagagcgacgcagggactgggggcgggcgggagggagccggggacggacggggggaggaaggcagggaggaaaagcggtcctcggcctccgggagtagcgggacccccgccctccgggaaaacggtcagcgtccggcgcgggctgagggctgggcccacagccgccgcgccggccggcgcggcacccattcgccccggttccgtggcccagggagtgggcggtttcctccgggacaaaagaccgggactcgggttgccgtcgggttttcacccgcgcggttcacagaccgcacatccccaggctgagccctgcaacgcggcgcgaggccgacagccccggccacggaggagccacacgcaggacgacggaggcgtgattttggtttccgcgtggctttgccctccgcaaggcggcctgttgctcacgtctctccggcccccgaaaggctggccatgccgactgtttgctcccggagctctgcgggcacccggaaacatgcagggaagggtgcaagcccggcatggtgccttcgctctccttgccaggttccaaaccggccacactgcagactccccacgttgccgcacgcgggaatccatcgtcaggccatcacgccggggaggcatctcctctctggggtctcgctctggtcttctacgtggaaatgaacgagagccacacgcctgcgtgtgcgagaccgtcccggcaacggcgacgcccacaggcattgcctccttcacggagagagggcctggcacactcaagactcccacggaggttcagttccacactcccctccaccctcccaggctggtttctccctgctgccgacgcgtgggagcccagagagcggcttcccgttcccgcgggatccctggagaggtccggagagccggcccccgaaacgcgcccccctcccccctcccccctctccccgttcctcttcgtctctccggccccaccaccaccaccgccaccacgccctcccccaccaccccccccccccccaccaccaccaccaccaccccgccggccggccccaggcctcgacgccctgggtcccttccggggtggggcgggctgtcccaggggggctcaccgccattcatgaaggggtggagcctgcctgcctgtgggcctttacaagggcggctggctgggtggctggctgtccgggcaggccccctggctgcacctgccgcagtgcacagtccggctgaggtgcacgggagcccgccggcctctctctgcccgcgtccgtccgtgaaattccggccggggctcaccgcgatggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttggcctacgccgccccggctcctccggacggggcgctctcccaccctcaggctcctcggtggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggttgggacggggtcgggtggttcggggcagggcggtggcctctctttcgcggggaacacctggctggctacggaggggcgtgtctccgccccgccccctccaccgggctgaccggcctgggattcctgccttctaggtctaggcccggtgagagactccacaccgcggagaactgccattctttcctgggcatcccggggatcccagagccggcccaggtaccagcaggtgggccgcctactgcgcacgcgcgggtttgcgggcagccgcctgggctgtgggagcagcccgggcagagctctcctgcctctccaccagcccaccccgccgcctgaccgccccctccccacccccaccccccacccccggaaaacgcgtcgtcccctgggctgggtggagacccccgtcccgcgaaacaccgggccccgcgcagcgtccgggcctgacaccgctccggcggctcgcctcctctgcgcccccgcgccaccgtcgcccgcccgcccgggcccctgcagccgcccaggtgccagcacggagcgcctggcggcggaacgcagaccccaggcccggcgcacaccggggacgctgagcgttccaggcgggagggaaggcgggcagagatggagagaggaacgggagacctagaggggcggaaggacgggcggagggacgttaggagggagggagggaggcagggaggcagggaggaacggagggaaagacagagcgacgcagggactgggggcgggcgggagggagccgggggacggggggaggaaggcagggaggaaaagcggtcctcggcctccgggagtagcgggacccccgccctccgggaaaacggtcagcgtccggcgcgggctgagggctgggcccacagccgccgcgccggccggcggggcaccacccattcgccccggttccggggcccagggagtgggcggtttcctccgggacaaaagaccgggactcgggttgccgtcgggtcttcacccgcgcggttcacagaccgcacatccccaggctcagccctgcaacgcggcgcgaggccgacagccccggccacggaggagccacacgcaggacgacggaggcgtgattttggtttccgcgtggctttgccctccgcaaggcggcctgttgctcacgtctctccggcccccgaaaggctggccatgccgactgtttgctcccggagctctgcgggcacccggaaacatgcagggaagggtgcaagcccggcacggtgccttcgctctccttgccaggttccaaaccggccacactgcagactccccacgttgccgcacgcgggaatccatcgtcaggccatcacgccggggaggcatctcctctctggggtctcgctctggtcttctacgtggaaatgaacgagagccacacgcctgcgtgtgcgagaccgtcccggcaacggcgacgcccacaggcattgcctccttcacggagagagggcctggcacactcaagactcccacggaggttcagttccacactcccctccaccctcccaggctggtttctccctgctgccgacgcgtgggagcccagagagcggcttcccgttcccgcgggatccctggagaggtccggagagccggcccccgaaacgcgcccccctcccccctcccccctctcccccttcctcttcgtctctccggccccaccaccaccaccgccaccacgccctccccccccaccccccccccccaccaccaccaccaccaccaccccgccggccggccccaggcctcgacgccctgggtcccttccggggtggggcgggctgtcccaggggggctcaccgccattcatgaaggggtggagcctgcctgcctgtgggcctttacaagggcggctggctggctggctggctgtccgggcaggcctcctggctgcacctgccgcagtgcacagtccggctgaggtgcacgggagcccgccggcctctctctgcccgcgtccgtccgtgaaattccggccggggctcaccgcgatggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcgctggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggttgggacggggtcgggtggttcggggcagggccgtggcctctctttcgcggggaacacctggctggctacggaggggcgtgtctccgccccgccccctccaccgggctgaccggcctgggattcctgccttctaggtctaggcccggtgagagactccacaccgcggagaactgccattctttcctgggcatcccggggatcccagagccggcccaggtaccagcaggtgggccgcctactgcgcacgcgcgggtttgcgggcagccgcctgggctgtgggagcagcccgggcagagctctcctgcctctccaccagcccaccccgccgcctgaccgccccctccccacccccaccccccacccccggaaaacgcgtcgtcccctgggctgggtggagacccccgtcccgcgaaacaccgggccccgcgcagcgtccgggcctgacaccgctccggcggctcgcctcctctgcgcccccgcgccaccgtcgcccgcccgcccgggcccctgcagcctcccagctgccagcgcggagctcctggcggtcaaaagcatacctctgtctgtctttgcccgcttcctggctagacctgcgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctatagaagatttgcatcttttgtgtgatgagtgcagagatatgtcacaatatcccctgtagaaaaagcctgaaattggtttacataacttcggtgatcagtgcagatgtgtttcagaactccatagtagactgaacctagagaatggttacatcacttaggtgatcagtgtagagatatgttaaaattctcgtgtagacagagcctagacaattgttacatcacctagtgatcagtgcagggataagtcataaagcctcctgtaggcagagtgtaggcaagtgttccctccctgggctgatcagtgcagagatatctcacaaagcccctataagccaaaccttgacaagggttacatcacctgtttgagcagtggaaatatatatcacaaagccccctgtagacaaagcccagacaatttttacatctcctgagtgagcattggagagatctgtcacaatgcccctgtaggcagagcttagacaagtgttacatcacctgggtgatcagtgcagagatatgtcaaaacgctcctgtagtctgaacctagacaggagttacatcaccttggggatcagtgcagagatacgtgagaattcc

SEQ ID NO: 67-70 - skipped (4 entries)

SEQ ID NO: 71 - DNA, 16 bp, synthetic construct
  source 1..16: mol_type = other DNA
gccggcccaggtacca

SEQ ID NO: 72 - DNA, 17 bp, synthetic construct
  source 1..17: mol_type = other DNA
cagcgagctcccttgca

SEQ ID NO: 73 - DNA, 14 bp, synthetic construct
  source 1..14: mol_type = other DNA
cagtgcgcaccccg

(19) United States
(12) Patent Application Publication
(10) Pub. No.: US 2025/0297251 A1
(43) Pub. Date: Sep. 25, 2025

(54) CERTAIN DUX4 INHIBITORS AND METHODS OF USE THEREOF

(71) Applicants: Ultragenyx Pharmaceutical Inc., Novato, CA (US); Saint Louis University, St. Louis, MI (US)

(72) Inventors: Sean Christopher DAUGHERTY, Petaluma, CA (US); Lishan CHEN, San Diego, CA (US); Francis Michael SVERDRUP, Lake St. Louis, MI (US)

(73) Assignees: Ultragenyx Pharmaceutical Inc., Novato, CA (US); Saint Louis University, St. Louis, MI (US)

(21) Appl. No.: 18/843,404

(22) PCT Filed: Feb. 27, 2023
(86) PCT No.: PCT/US2023/063334

§ 371 Date: Sep. 3, 2024

Related U.S. Application Data
(60) Provisional application No. 63/315,611, filed on Mar. 2, 2022.

(51) Int. Cl. C12N 15/113; A61K 47/54; A61K 47/68

(52) U.S. Cl. CPC C12N 15/113; A61K 47/542; A61K 47/6807; A61K 47/6843; C12N 2310/14; C12N 2310/315; C12N 2310/321; C12N 2310/322; C12N 2310/3231; C12N 2310/344; C12N 2310/3513; C12N 2310/3515

(57) ABSTRACT

This application relates to double-stranded small interfering RNAs that modulate DUX4 gene expression and describes methods of inhibiting DUX4 gene expression by contacting a cell with said double-stranded small interfering RNAs. The application further provides compositions comprising said double-stranded small interfering RNAs and their use in methods of preventing or treating a disease or disorder associated with aberrant expression of DUX4, such as facioscapulohumeral muscular dystrophy (FSHD) or cancer, in a subject.

### CROSS-REFERENCE TO RELATED APPLICATIONS

This application claims the benefit of priority under 35 U.S.C. 119(e) to U.S. provisional application No. 63/315,611, filed Mar. 2, 2022, the contents of which are hereby incorporated by reference in its entirety.

### REFERENCE TO A SEQUENCE LISTING

This application contains a Sequence Listing submitted electronically as an XML file. The text file, named “8957-7-PCT_Sequence_listing.xml”, has a size in bytes of 337,000 bytes and was prepared on Feb. 27, 2023. The information contained in the XML file is incorporated herein by reference in its entirety pursuant to 37 CFR § 1.52(e)(5).

### TECHNICAL FIELD OF THE DISCLOSURE

This invention relates to double-stranded small interfering RNAs (siRNAs) that modulate DUX4 gene expression, and their applications in research, diagnostics, and/or therapeutics. In some embodiments, it relates to compositions and methods comprising the said siRNAs in the prevention and/or treatment of facioscapulohumeral muscular dystrophy (FSHD).

### BACKGROUND OF THE INVENTION

Facioscapulohumeral muscular dystrophy (FSHD) is a rare genetic disease affecting about one in 10,000 people worldwide. FSHD patients exhibit progressive, asymmetric muscle weakness and up to 20% of affected individuals become severely disabled. Non-muscular symptoms include subclinical sensorineural hearing loss telangiectasia.

Aberrant expression of the DUX4 protein in skeletal muscle due to inefficient epigenetic repression of the DUX4 gene is thought to cause FSHD. DUX4 is a retrogene encoded in each unit of the D4Z4 macrosatellite repeat array. D4Z4 repeats are bi-directionally transcribed in somatic tissues and generate long stretches of RNA and small RNA fragments that may have a role in epigenetic silencing. The more prevalent form of FSHD (FSHD1) is caused by the deletion of a subset of D4Z4 macrosatellite repeats in the subtelomeric region of chromosome 4q. Unaffected individuals have 11-100 of the 3.3 kb D4Z4 repeat units, whereas FSHD1 individuals have 10 or fewer repeats. FSHD2 is associated with decreased DNA methylation of the D4Z4 repeats on the same 4qA haplotype. Thus, administration of agents that suppress expression of the DUX4 gene is a promising therapeutic approach for preventing or treating FSHD1 and FSHD2. Beyond their potential utility in the prevention or treatment of FSHD, DUX4-targeted treatments may also improve the success of cancer immunotherapies, as DUX4 expression been found to suppress MHC class I to promote cancer immune evasion and mediate resistance to anti-CTLA-4 therapy. See Chew et al., 2019, Dev Cell 50(5): 525-6.

Double-stranded oligonucleotides have been used to modulate gene expression for use in research, diagnostics, and/or therapeutics. One method of modulation of gene expression is RNA interference (RNAi), which generally refers to gene silencing involving the introduction of double-stranded RNA (dsRNA) leading to the sequence-specific reduction of targeted endogenous mRNA levels. The reduction of target mRNA may occur by one of several different mechanisms, depending on the sequence or structure of the dsRNA. For example, it may lead to degradation of the target mRNA through formation of RNA induced silencing complex (RISC), or transcriptional silencing in which transcription of the mRNA is inhibited in a process called RNA-induced transcriptional silencing (RITS), or by modulation of microRNA (miRNA) function. MicroRNAs are small non-coding RNAs that regulate the expression of messenger RNAs. The binding of an RNAi compound to a microRNA prevents that microRNA from binding to its messenger RNA targets, and thus interferes with the function of the microRNA. The sequence-specificity of RNAi compounds makes them promising candidates as therapeutics to selectively modulate the expression of genes involved in the pathogenesis of diseases.

There continues to be a need in the art for methods and agents for treatment of FSHD. The present application addresses this need by providing oligonucleotides, and compositions and methods comprising them, that can suppress aberrant expression of DUX4 gene and thus can ameliorate, prevent or treat FSHD.

### BRIEF SUMMARY OF THE INVENTION

The present invention provides double-stranded small interfering RNA (siRNA) molecules for reducing or inhibiting the expression of the DUX4 gene. The present invention also provides a method of reducing or inhibiting expression of DUX4 in a cell comprising contacting the cell with a double-stranded siRNA molecule targeted to DUX4, thereby reducing or inhibiting expression of DUX4. In another aspect, the invention provides compositions and methods for the prevention or treatment of various disorders, including facioscapulohumeral muscular dystrophy (FSHD) by administering the double-stranded siRNA molecules and compositions comprising the same to a subject.

Accordingly, in one aspect, the present invention includes double-stranded small interfering RNA (siRNA) molecules, each molecule comprising a sense strand and an antisense strand, that are useful for reducing or inhibiting the aberrant expression of the DUX4 gene in a cell. In some embodiments, the double-stranded small interfering RNA comprises at least one modified nucleoside.

In some embodiments, the DUX4 gene comprises a nucleobase sequence that is at least 85%, at least 90% identical, or at least 95% identical to SEQ ID NO: 66. In certain embodiments, the DUX4 gene comprises a nucleobase sequence that is 100% identical to SEQ ID NO: 66.

In some embodiments, the antisense strand of the double-stranded small interfering RNA comprises a nucleobase sequence comprising any of the nucleobase sequences listed in Table 1, i.e., a sequence selected from the group consisting of SEQ ID NOs: 2, 4, 6, 8, 10, 12, 14, 16, 18, 20, 22, 24, 26, 28, 30, and 32.

In some embodiments, the sense strand of the double-stranded small interfering RNA comprises a nucleobase sequence comprising any one of the nucleobase sequences listed in Table 1, i.e., a sequence selected from the group consisting of SEQ ID NOs: 1, 3, 5, 7, 9, 11, 13, 15, 17, 19, 21, 23, 25, 27, 29, and 31.

In some embodiments, the double-stranded small interfering RNA comprises at least one modified nucleoside. In some embodiments, the sense strand of the double-stranded small interfering RNA comprises at least one modified nucleoside. In some embodiments, each nucleoside of the sense strand of the double-stranded small interfering RNA comprises a modified nucleoside.

In some embodiments, at least one nucleoside of the sense strand of the double-stranded siRNA comprises a modified sugar. In some embodiments, each nucleoside of the sense strand of the double-stranded siRNA comprises a modified sugar. In some embodiments, the modified nucleoside comprises a 2′-F modified sugar and/or a 2′-OMe modified sugar. In some embodiments, the modified nucleoside comprises a 2′-OMe modified sugar. In some embodiments, the modified nucleoside comprises a 2′-F modified sugar modified sugar. In some embodiments, the antisense and/or the sense strand of the double-stranded small interfering RNA comprises a TT overhang at the 3′ end.

In some embodiments, the sense strand of the double-stranded small interfering RNA comprises at least one modified internucleoside linkage. In some embodiments, the sense strand of the double-stranded small interfering RNA comprises at least 2, 3, 4, or 5 modified internucleoside linkages. In some embodiments, the modified internucleoside linkage is a phosphorothioate linkage.

In some embodiments, the double-stranded small interfering RNA comprises any one of the modified nucleobase sequences shown in Table 2. In some embodiments, the double-stranded siRNA comprises a sense strand and an antisense strand wherein the sense strand comprises a nucleotide sequence selected from the group consisting of SEQ ID NOs: 46, 52, 56, 33, 35, 37, 39, 41, 42, 44, 48, 50, 54, 58, 60, 62 and 64; and wherein the antisense strand comprises a nucleotide sequence selected from the group consisting of SEQ ID NOs: 47, 53, 57, 34, 36, 38, 40, 43, 45, 49, 51, 55, 59, 61, 63 and 65.

In some embodiments, the antisense strand of the double-stranded small interfering RNA comprises a nucleobase sequence comprising any of the nucleobase sequences listed in Table 2, i.e., a sequence selected from the group consisting of SEQ ID NOs: 47, 53, 57, 34, 36, 38, 40, 43, 45, 49, 51, 55, 59, 61, 63 and 65.

In some embodiments, the sense strand of the double-stranded small interfering RNA comprises a nucleobase sequence comprising any of the nucleobase sequences listed in Table 2, i.e., a sequence selected from the group consisting of SEQ ID NOs: 46, 52, 56, 33, 35, 37, 39, 41, 42, 44, 48, 50, 54, 58, 60, 62 and 64.

In some embodiments, the double-stranded small interfering RNA is conjugated to a lipophilic molecule, an antibody, an aptamer, a ligand, a peptide, or a polymer. In some embodiments, the lipophilic molecule may be a long chain fatty acid (LCFA). In some embodiments, the antibody is an anti-transferrin receptor antibody.

In another aspect, the present invention includes a pharmaceutical composition comprising a double-stranded small interfering RNA described herein or a salt thereof, and at least one pharmaceutically acceptable carrier. The pharmaceutical composition may be for use in medical therapy. The pharmaceutical composition may be for use in in the treatment of a human or animal body. In another aspect, the present invention includes a use of the pharmaceutical composition for preparing or manufacturing a medicament for ameliorating, preventing, delaying onset, or treating a disease or disorder associated with aberrant expression of DUX4 in a subject in need thereof. In another aspect, the present invention includes a method for ameliorating, preventing, delaying onset, or treating a disease or disorder associated with aberrant expression of DUX4 in a subject in need thereof by administering the pharmaceutical composition to the subject. The disease or disorder may be facioscapulohumeral muscular dystrophy (FSHD) and may be FSHD1 or FSHD2. In another aspect, the present invention includes a method of ameliorating, preventing, delaying onset, or treating facioscapulohumeral muscular dystrophy (which includes FSHD1 and FSHD2) by administering the pharmaceutical composition to the subject.

In various embodiments, the administration may be intravenous, subcutaneous, pulmonary, intramuscular, intraperitoneal, dermal, oral, nasal, or via inhalation. In some embodiments, the administration may be once daily, weekly, every two weeks, monthly, every two months, quarterly, or yearly. In some embodiments, the administration may comprise an effective dose of from 0.01 to 100 mg/kg. In some embodiments, the administration inhibits the expression of DUX4 in the subject.

In another aspect, the invention comprises a kit comprising one or more double-stranded siRNA and a device for administering said double-stranded siRNA.

In another aspect, the present invention includes a method of ameliorating, preventing, delaying onset, or treating facioscapulohumeral muscular dystrophy (which includes FSHD1 and FSHD2) comprising administering a double-stranded small interfering RNA described herein. In another aspect, the present invention includes use of a double-stranded small interfering RNA described herein for the treatment of facioscapulohumeral muscular dystrophy.

In another aspect, the present invention includes a method of ameliorating, preventing, delaying onset, or treating cancer, comprising administering a double-stranded small interfering RNA described herein. In some embodiments, the method may further comprise the administration of a checkpoint inhibitor such as an anti-CTLA-4 agent.

In another aspect, the present invention includes use of a double-stranded small interfering RNA described herein for the preparation of a medicament for the treatment of facioscapulohumeral muscular dystrophy.

In another aspect, the present invention includes a method of inhibiting expression of DUX4 in a cell, comprising contacting a cell with a double-stranded small interfering RNA described here, and thereby inhibiting expression of DUX4. In some embodiments, the contacting is performed in vitro. In some embodiments, the contacting is performed in vivo. In some embodiments, the cell is in an animal. In some embodiments, the animal is a human. In some embodiments, the expression of DUX4 is inhibited by at least 10%, at least 20%, at least 30%, at least 40%, at least 50%, at least 60%, at least 70%, at least 80%, or at least 90%. In some embodiments, the expression of DUX4 is abolished.

These and other aspects and features of the disclosure are described in the following sections of the application.

### DETAILED DESCRIPTION OF THE INVENTION

Described herein are double-stranded small interfering RNA (siRNA) molecules that target sequences within the DUX4 gene. Also described are methods of reducing or inhibiting expression of DUX4 in a cell comprising contacting the cell with the said siRNA molecules.

Further described herein are methods for the prevention or treatment of facioscapulohumeral muscular dystrophy (FSHD) by administering to a subject the double-stranded siRNA molecules and compositions comprising the same.

### Definitions

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

As used herein, the term “DUX4” may refer to a DUX4 protein or a DUX4 nucleic acid, i.e., a nucleic acid sequence encoding a DUX4 protein. A DUX4 nucleic acid may refer to a DNA sequence encoding DUX4 protein, an RNA sequence transcribed from DNA encoding DUX4 (including genomic DNA comprising introns and exons) including a non-protein encoding (i.e., non-coding) RNA sequence, and an mRNA sequence encoding DUX4.

In some embodiments, DUX4 nucleic acid sequence comprises GENBANK Accession No. FJ439133.1 (SEQ ID NO: 66).

“Double-stranded small interfering RNA” means any duplex RNA structure comprising two anti-parallel and substantially complementary nucleic acid strands. In certain embodiments, double-stranded small interfering RNA comprise a sense strand and an antisense strand, wherein the antisense strand is complementary to a target nucleic acid.

“Complementarity” means the capacity for pairing between nucleobases of a first nucleic acid and a second nucleic acid.

“Contiguous nucleobases” means nucleobases immediately adjacent to each other.

“Deoxyribonucleotide” means a nucleotide having a hydrogen at the 2′ position of the sugar portion of the nucleotide. Deoxyribonucleotides may be modified with any of a variety of substituents.

“Expression” includes all the functions by which a gene's coded information is converted into structures present and operating in a cell. Such structures include, but are not limited to, the products of transcription and translation.

“Fully complementary” or “100% complementary” means each nucleobase of a first nucleic acid has a complementary nucleobase in a second nucleic acid. In certain embodiments, a first nucleic acid is an antisense compound, and a target nucleic acid is a second nucleic acid.

“Inhibiting the expression or activity” refers to a reduction or blockade of the expression or activity and does not necessarily indicate a total elimination of expression or activity.

“Internucleoside linkage” refers to the chemical bond between nucleosides.

“Linked nucleosides” means adjacent nucleosides linked together by an internucleoside linkage.

“Modified internucleoside linkage” refers to a substitution or any change from a naturally occurring internucleoside bond (i.e., a phosphodiester internucleoside bond).

“Nucleobase” means a heterocyclic moiety capable of pairing with a base of another nucleic acid. “Modified nucleobase” means any nucleobase other than adenine, cytosine, guanine, thymidine, or uracil. An “unmodified nucleobase” means the purine bases adenine (A) and guanine (G), and the pyrimidine bases thymine (T), cytosine (C) and uracil (U).

“Nucleoside” means a nucleobase linked to a sugar. “Modified nucleoside” means a nucleoside having, independently, a modified sugar moiety and/or modified nucleobase.

“Modified nucleotide” means a nucleotide having, independently, a modified sugar moiety, modified internucleoside linkage, or modified nucleobase.

Modified sugar” means substitution and/or any change from a natural sugar moiety. In certain embodiments modified sugars include 2′-F modified sugars and 2′-OMe modified sugars.

“Nucleobase complementarity” refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

“Nucleobase sequence” means the order of contiguous nucleobases independent of any sugar, linkage, and/or nucleobase modification.

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Sites,” as used herein, are defined as unique nucleobase positions within a target nucleic acid.

“Subject” means a human or non-human animal selected for treatment or therapy.

“Target gene” refers to a gene encoding a target.

“Target nucleic acid” refers to a nucleic acid, the modulation of which is desired.

Double-Stranded Small Interfering RNA (siRNA) Molecules

In one aspect, the present invention includes double-stranded oligonucleotides, such as small interfering RNA (siRNA) compounds, and compositions comprising the same. In some embodiments, siRNA compounds may comprise at least one modified RNA nucleoside (i.e., independently, a modified sugar moiety and/or modified nucleobase), and/or modified internucleoside linkages. In certain embodiments, siRNA compounds may comprise modified RNA nucleosides, modified DNA nucleosides, and/or modified internucleoside linkages.

Some embodiments relate to double-stranded molecules wherein each strand comprises a motif defined by the location of one or more modified or unmodified nucleosides.

In some embodiments, compositions are provided comprising a first and a second oligomeric compound that are fully or at least partially hybridized to form a duplex region and further comprising a region that is complementary to and hybridizes to a nucleic acid target. Such a composition may comprise a first oligomeric compound that is an antisense strand having full or partial complementarity to a nucleic acid target and a second oligomeric compound that is a sense strand having one or more regions of complementarity to and forming at least one duplex region with the first oligomeric compound.

In some embodiments, the compositions of the present invention modulate gene expression by hybridizing to a nucleic acid target resulting in loss of its normal function. In some embodiments, the degradation of the target nucleic acid is facilitated by an activated RISC complex that is formed with compositions of the invention.

In some embodiments, one of the strands is useful in, for example, influencing the preferential loading of the opposite strand into the RISC (or cleavage) complex. In some embodiments, the compositions of the present invention hybridize to a portion of a target RNA resulting in loss of normal function of the target RNA.

Some embodiments are drawn to double-stranded compositions wherein both the strands comprise a hemimer motif, a fully modified motif, a positionally modified motif or an alternating motif. Each strand of the compositions of the present invention may be modified to fulfill a particular role in for example the siRNA pathway. Using a different motif in each strand or the same motif with different chemical modifications in each strand permits targeting the antisense strand for the RISC complex while inhibiting the incorporation of the sense strand. Within this model, each strand may be independently modified such that it is enhanced for its particular role. The antisense strand may be modified at the 5′-end to enhance its role in one region of the RISC while the 3′-end may be modified differentially to enhance its role in a different region of the RISC.

The double-stranded oligonucleotide molecules may comprise self-complementary sense and antisense regions, wherein the antisense region comprises a nucleotide sequence that is complementary to nucleotide sequence in a target nucleic acid molecule or a portion thereof and the sense region having nucleotide sequence corresponding to the target nucleic acid sequence or a portion thereof. The double-stranded oligonucleotide molecules can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double-stranded structure, for example wherein the double-stranded region is about 12 to about 30, e.g., about 12, 13, 14, 15, 16, 17, 18, 19, 20, 21, 22, 23, 24, 25, 26, 27, 28, 29 or 30 base pairs; the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof (e.g., about 12 to about 30 or more nucleotides of the double-stranded oligonucleotide molecule are complementary to the target nucleic acid or a portion thereof). Alternatively, the double-stranded oligonucleotide may be assembled from a single oligonucleotide, where the self-complementary sense and antisense regions of the siRNA are linked by means of a nucleic acid based or non-nucleic acid-based linker(s).

The double-stranded oligonucleotide may have a duplex, asymmetric duplex, hairpin or asymmetric hairpin secondary structure, having self-complementary sense and antisense regions, wherein the antisense region comprises a nucleotide sequence that is complementary to a nucleotide sequence in a separate target nucleic acid molecule or a portion thereof and the sense region having a nucleotide sequence corresponding to the target nucleic acid sequence or a portion thereof. The double-stranded oligonucleotide can be a circular single-stranded polynucleotide having two or more loop structures and a stem comprising self-complementary sense and antisense regions, wherein the antisense region comprises a nucleotide sequence that is complementary to a nucleotide sequence in a target nucleic acid molecule or a portion thereof and the sense region having a nucleotide sequence corresponding to the target nucleic acid sequence or a portion thereof, and wherein the circular polynucleotide can be processed either in vivo or in vitro to generate an active siRNA molecule capable of mediating RNAi.

In some embodiments, the double-stranded oligonucleotide comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der waals interactions, hydrophobic interactions, and/or stacking interactions. In some embodiments, the double-stranded oligonucleotide comprises a nucleotide sequence that is complementary to a nucleotide sequence of a target gene. In another embodiment, the double-stranded oligonucleotide interacts with a nucleotide sequence of a target gene in a manner that causes inhibition of expression of the target gene.

As used herein, the term siRNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics. For example, double-stranded oligonucleotides can be used to epigenetically silence genes at both the post-transcriptional level and the pre-transcriptional level. In a non-limiting example, epigenetic regulation of gene expression by siRNA molecules of the invention can result from siRNA mediated modification of chromatin structure or methylation pattern to alter gene expression (see, for example, Verdel et a., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237).

It is contemplated that compounds and compositions of some embodiments provided herein can target by a dsRNA-mediated gene silencing or RNAi mechanism, including, e.g., “hairpin” or stem-loop double-stranded RNA effector molecules in which a single RNA strand with self-complementary sequences is capable of assuming a double-stranded conformation, or duplex dsRNA effector molecules comprising two separate strands of RNA. In various embodiments, the dsRNA consists entirely of ribonucleotides or consists of a mixture of ribonucleotides and deoxynucleotides, such as the RNA/DNA hybrids disclosed, for example, by WO 00/63364, filed Apr. 19, 2000, or U.S. Ser. No. 60/130,377, filed Apr. 21, 1999.

As used herein, double-stranded oligonucleotides need not be limited to those molecules containing only RNA, but further encompasses chemically modified nucleotides and non-nucleotides. In certain embodiments, the short interfering nucleic acid molecules may lack ribonucleotides or 2′-hydroxy (2′-OH) containing nucleotides. Such double-stranded oligonucleotides that do not require the presence of ribonucleotides within the molecule to support RNAi can however have an attached linker or linkers or other attached or associated groups, moieties, or chains containing one or more nucleotides with 2′-OH groups. In some embodiments, the double-stranded oligonucleotides can comprise ribonucleotides at about 5, 10, 20, 30, 40, or 50% of the nucleotide positions.

The dsRNA or dsRNA effector molecule may be a single molecule with a region of self-complementarity such that nucleotides in one segment of the molecule base pair with nucleotides in another segment of the molecule. In various embodiments, a dsRNA that consists of a single molecule consists entirely of ribonucleotides or includes a region of ribonucleotides that is complementary to a region of deoxyribonucleotides. Alternatively, the dsRNA may include two different strands that have a region of complementarity to each other.

In various embodiments, both strands consist entirely of ribonucleotides, one strand consists entirely of ribonucleotides and one strand consists entirely of deoxyribonucleotides, or one or both strands contain a mixture of ribonucleotides and deoxyribonucleotides.

In certain embodiments, the regions of complementarity are at least 70, 80, 90, 95, 98, or 100% complementary to each other and to a target nucleic acid sequence. In certain embodiments, the region of the dsRNA that is present in a double-stranded conformation includes at least 6, 7, 8, 9, 10, 11, 12, 13, 14, 15, 16, 17, 18, 19, 20, 21, 22, 23, 24, 25, 26, 27, 28, 29, 30, 50, 75, 100, 200, 500, 1000, 2000 or 5000 nucleotides or includes all of the nucleotides in a cDNA or other target nucleic acid sequence being represented in the dsRNA. In some embodiments, the dsRNA does not contain any single stranded regions, such as single stranded ends, or the dsRNA is a hairpin. In other embodiments, the dsRNA has one or more single stranded regions or overhangs. In certain embodiments, RNA/DNA hybrids include a DNA strand or region that is an antisense strand or region (e.g., has at least 70, 80, 90, 95, 98, or 100% complementarity to a target nucleic acid) and an RNA strand or region that is a sense strand or region (e.g., has at least 70, 80, 90, 95, 98, or 100% identity to a target nucleic acid), and vice versa.

In various embodiments, the RNA/DNA hybrid is made in vitro using enzymatic or chemical synthetic methods such as those described herein or those described in WO 00/63364, filed Apr. 19, 2000, or U.S. Ser. No. 60/130,377, filed Apr. 21, 1999. In other embodiments, a DNA strand synthesized in vitro is complexed with an RNA strand made in vivo or in vitro before, after, or concurrent with the transformation of the DNA strand into the cell.

In yet other embodiments, the dsRNA is a single circular nucleic acid containing a sense and an antisense region, or the dsRNA includes a circular nucleic acid and either a second circular nucleic acid or a linear nucleic acid (see, for example, WO 00/63364, filed Apr. 19, 2000, or U.S. Ser. No. 60/130,377, filed Apr. 21, 1999). Exemplary circular nucleic acids include lariat structures in which the free 5′ phosphoryl group of a nucleotide becomes linked to the 2′ hydroxyl group of another nucleotide in a loop back fashion. Chemically synthesized RNA duplexes in the 25-30 base length range have been shown to exhibit increased potency compared with shorter 21-mer siRNAs and the increase in potency is attributed to the action of Dicer endonuclease enzyme which uses the longer dsRNA as substrate, cleaves it and facilitates the loading of the cleaved dsRNA into the RISC. Thus, in some embodiments, the dsRNA may be a dicer substrate RNA. For example, in some embodiments, the dsRNA is a 25/27 mer.

Modified Nucleotides and Chemically Modified siRNAs

In various embodiments described herein, a double-stranded siRNA of the invention may comprise one or more (e.g., two, three, four, five, or more) modified nucleic acid monomers (i.e., nucleotides). Various examples of modified nucleotides are disclosed in U.S. Pat. Nos. 9,035,039, 9,951,338, 10,036,024, 10,538,763, and International Patent Publication No. WO/2018/222926, each of which is herein incorporated by reference in its entirety.

Examples of modified nucleotides suitable for use in the present invention include, but are not limited to, ribonucleotides or arabinonucleotides having a 2′-O-methyl (2′-OMe), 2′-deoxy-2′-fluoro (2′-F), 2′-deoxy, 5-C-methyl, 2′-O-(2-methoxyethyl) (MOE), 4′-thio, 2′-amino, or a 2′-C-allyl group. In some embodiments, these may include 2′-deoxy-2′-fluoro arabinoguanosine nucleotides.

Modified nucleotides having a conformation such as those described in the art, for example in Sanger, Principles of Nucleic Acid Structure, Springer-Verlag Ed. (1984), are also suitable for use in siRNA molecules. Other modified nucleotides include, without limitation, locked nucleic acid (LNA) nucleotides, unlocked nucleic acid (UNA) nucleotides, G-clamp nucleotides, or nucleotide base analogs. LNA nucleotides include but need not be limited to 2′-O, 4′-C-methylene-(D-ribofuranosyl)nucleotides), 2′-O-(2-methoxyethyl) (MOE) nucleotides, 2′-methyl-thio-ethyl nucleotides, 2′-deoxy-2′-fluoro (2′-F) nucleotides, 2′-deoxy-2′-chloro (2′-Cl) nucleotides, 2′-azido nucleotides, and (S)-constrained ethyl (cEt) nucleotides.

In some embodiments, a double-stranded siRNA molecule may comprise one or more chemical modifications such as terminal cap moieties, phosphate backbone modifications, and the like. Examples of classes of terminal cap moieties include, without limitation, inverted deoxy abasic residues, glyceryl modifications, 4′,5′-methylene nucleotides, 1-(beta-D-erythrofuranosyl) nucleotides, 4′-thio nucleotides, carbocyclic nucleotides, 1,5-anhydrohexitol nucleotides, L-nucleotides, alpha-nucleotides, modified base nucleotides, threo pentofuranosyl nucleotides, acyclic 3′,4′-seco nucleotides, acyclic 3,4-dihydroxybutyl nucleotides, acyclic 3,5-dihydroxypentyl nucleotides, 3′-3′-inverted nucleotide moieties, 3′-3′-inverted abasic moieties, 3′-2′-inverted nucleotide moieties, 3′-2′-inverted abasic moieties, 5′-5′-inverted nucleotide moieties, 5′-5′-inverted abasic moieties, 3′-5′-inverted deoxy abasic moieties, 5′-amino-alkyl phosphate, 1,3-diamino-2-propyl phosphate, 3 aminopropyl phosphate, 6-aminohexyl phosphate, 1,2-aminododecyl phosphate, hydroxypropyl phosphate, 1,4-butanediol phosphate, 3′-phosphoramidate, 5′ phosphoramidate, hexylphosphate, aminohexyl phosphate, 3′-phosphate, 5′-amino, 3′-phosphorothioate, 5′-phosphorothioate, phosphorodithioate, and bridging or non-bridging methylphosphonate or 5′-mercapto moieties. Non-limiting examples of phosphate backbone modifications (i.e., resulting in modified internucleoside linkages) include phosphorothioate, phosphorodithioate, methylphosphonate, phosphotriester, morpholino, amidate, carbamate, carboxymethyl, acetamidate, polyamide, sulfonate, sulfonamide, sulfamate, formacetal, thioformacetal, and alkylsilyl substitutions. Such chemical modifications can occur at the 5′-end and/or 3′-end of the sense strand, antisense strand, or both strands of the siRNA.

In some embodiments, a double-stranded siRNA of the invention may comprise at least one modified internucleoside linkage. Examples of modified internucleoside linkage include, without limitation, peptide linkage, phosphorothioate (PS) linkage, and phosphorodiamidate morpholino (PMO) linkage. In some embodiments, a double-stranded siRNA of the invention comprises 2, 3, 4, 5, or more modified internucleoside linkages. In some embodiments, a double-stranded siRNA of the invention comprises a sense strand, an antisense strand, or both, where all internucleoside linkages are modified. In some embodiments, each internucleoside linkage is a phosphorothioate internucleoside linkage.

In some embodiments, the sense and/or antisense strand may comprise a 5′-terminal or 3′-terminal overhang having 1, 2, 3, 4, or more 2′-deoxyribonucleotides (e.g., A, G, C, or T) and/or any combination of modified and unmodified nucleotides. In some embodiments, a double-stranded siRNA may comprise a TT overhang at the 3′ end of the sense strand. In some embodiments, a double-stranded siRNA may comprise a TT overhang at the 3′ end of the antisense strand. In some exemplary embodiments, a double-stranded siRNA may comprise a TT overhang at the 3′ end of sense strand and at the 3′ end of the antisense strand.

Conjugated siRNAs

In some embodiments, a double-stranded siRNA of the invention may be conjugated to at least one other molecule. Conjugation of the double-stranded siRNA with an appropriate molecule provides a means for improving delivery of the siRNA into target cells. Such conjugate molecules may interact with the lipid components of the cell membrane, bind to specific cell surface proteins or receptors, and/or penetrate the cell through endogenous transport mechanisms carrying the siRNA with them.

The conjugate can be attached at the 5′- and/or the 3′-end of the sense and/or the antisense strand of the siRNA via a covalent attachment such as a nucleic acid or non-nucleic acid linker. The conjugate can be attached to the siRNA through a carbamate group or other linking group (see, e.g., U.S. Patent Publication Nos. 20050074771, 20050043219, and 20050158727). A conjugate may be added to siRNA for any of a number of purposes. For example, the conjugate may be a molecular entity that facilitates the delivery of siRNA into a cell or may be a molecule that comprises a drug or label. Examples of conjugate molecules suitable for attachment to siRNA of the present invention include, without limitation, lipophilic molecules (e.g., fatty acids), cholesterols, glycols such as polyethylene glycol (PEG), human serum albumin (HSA), carotenoids, terpenes, bile acids, folates (e.g., folic acid, folate analogs and derivatives thereof), sugars (e.g., galactose, galactosamine, N-acetyl galactosamine, glucose, mannose, fructose, fucose, etc.), phospholipids, peptides, ligands for cellular receptors capable of mediating cellular uptake, antibodies, aptamers, and combinations thereof (see, e.g., U.S. Patent Publication Nos. 20030130186, 20040110296, and 20040249178; U.S. Pat. No. 6,753,423).

The type of conjugate used and the extent of conjugation to the siRNA can be evaluated for improved pharmacokinetic profiles, bioavailability, and/or stability of the siRNA while retaining activity. As such, one skilled in the art can screen siRNA molecules having various conjugates attached thereto to identify siRNA conjugates having improved properties using any of a variety of well-known in vitro cell culture or in vivo animal models including the negative-controlled expression studies described above. Examples of siRNA bioconjugates are described in, e.g., Chernikov et al., 2019, Front. Pharmacol. 10: 1-25 and Osborn et al., 2018, Nuc. Acid Ther. 28(3): 128-136.

In some embodiments, a double-stranded siRNA of the invention may be conjugated to a lipophilic molecule (for example, a long chain fatty acid or LCFA), an antibody (for example, anti-transferrin receptor antibody), an aptamer, a ligand, a peptide, or a polymer.

In one embodiment, the double-stranded siRNA may be conjugated to a lipophilic molecule, e.g., a long chain fatty acid. In some exemplary embodiments, a double-stranded siRNA of the invention is conjugated to a long chain fatty acid described in International Patent Publication No. WO/2019/232255.

In some embodiments, a double-stranded siRNA of the invention may be conjugated to an antibody. In some embodiments, the antibody is a muscle-targeting antibody. In some embodiments, the muscle-targeting antibody is an anti-transferrin receptor antibody. In some exemplary embodiments, a double-stranded siRNA of the invention is conjugated to an anti-transferrin receptor antibody described in International Patent Publication No. WO/2020/028864.

siRNA Delivery with Liposomes, Lipid Nanoparticles (LNPs), and Other Carriers

In some embodiments, a double-stranded siRNA of the invention may be delivered via liposomes, nanoparticles, lipid nanoparticles (LNPs), polymers, microparticles, microcapsules, micelles, or extracellular vesicles.

In some embodiments, a double-stranded siRNA of the invention is delivered via a lipid nanoparticle (LNP). Examples of LNPs capable of delivering a double-stranded siRNA of the invention are described in International Patent Publication Nos. WO/2015/074085, WO/2016/081029, WO/2017/117530, WO/2018/118102, WO/2018/119163, WO/2018/222926, WO/2019/191780, and WO/2020/154746. In some embodiments, an LNP may be decorated with targeting moiety, e.g., an antibody, a receptor, or a fragment thereof capable of binding to a target ligand.

In one embodiment, a lipid nanoparticle for use in the instant invention comprises (a) a nucleic acid (e.g., a double-stranded siRNA), (b) a cationic lipid, (c) an aggregation reducing agent (such as a PEG-lipid), (d) optionally a non-cationic lipid (such as a neutral lipid), and (e) optionally a sterol. In one embodiment, the lipid nanoparticle comprises (i) at least one cationic lipid; (ii) a neutral lipid, e.g., DSPC; (iii) a sterol, e.g., cholesterol; and (iv) a PEG-lipid, in a molar ratio of about 20-65% cationic lipid: 5-25% neutral lipid: 25-55% sterol; 0.5-15% PEG-lipid. In some embodiments, the cationic lipid is selected from ATX-002, ATX-081, ATX-095, or ATX-126, as described in WO/2018/222926.

In some embodiments, a double-stranded siRNA of the invention is delivered via a nanocarrier comprising a molecule enabling specific receptor-mediated endosomal uptake. In one embodiment, said molecule can enable receptor binding, endosomal uptake, controlled breakdown of the endosomal membrane, and release of siRNA into a target cell. Examples of nanocarriers capable of delivering a double-stranded siRNA of the invention are described in WO/2009/141257. In some embodiments, the nanocarrier is a lipid-based nanocarrier, e.g., a lipid nanoparticle (LNP).

### DUX4 Inhibitors

In another aspect, the present invention includes a method of reducing expression of DUX4 in a cell comprising contacting the cell with a double-stranded small interfering RNA compound targeted to DUX4. In certain embodiments, DUX4 comprises a nucleic acid sequence at least 85% identical to SEQ ID NO: 66. In certain embodiments, DUX4 comprises a nucleic acid sequence at least 85% complementary to SEQ ID NO: 66.

The inefficient epigenetic repression of DUX4 in skeletal muscle leads to aberrant expression of the DUX4 protein and facioscapulohumeral muscular dystrophy (FSHD) 1 and 2. FSHD1 and 2 patients exhibit progressive, asymmetric muscle weakness. Therefore, in certain embodiments it is desirable to inhibit expression of DUX4. In certain embodiments it is desirable to inhibit expression of DUX4 in a subject having 10 or fewer D4Z4 repeats.

In certain embodiments, DUX4 expression is inhibited by contacting a cell with a double-stranded small interfering RNA compound. In certain embodiments, DUX4 expression is inhibited by contacting a cell with a double-stranded small interfering RNA compound disclosed herein.

### Pharmaceutical Compositions

In certain embodiments, the present invention provides pharmaceutical compositions comprising one or more the double-stranded small interfering RNA compounds. In certain embodiments, such pharmaceutical composition comprises a suitable pharmaceutically acceptable diluent or carrier. In certain embodiments, a pharmaceutical composition comprises a sterile saline solution and one or more antisense compound. In certain embodiments, such pharmaceutical composition consists of a sterile saline solution and one or more antisense compound. In certain embodiments, the sterile saline is pharmaceutical grade saline. In certain embodiments, a pharmaceutical composition comprises one or more antisense compound and sterile water. In certain embodiments, a pharmaceutical composition consists of one or more antisense compound and sterile water. In certain embodiments, the sterile saline is pharmaceutical grade water. In certain embodiments, a pharmaceutical composition comprises one or more antisense compound and phosphate-buffered saline (PBS). In certain embodiments, a pharmaceutical composition consists of one or more antisense compound and sterile phosphate-buffered saline (PBS). In certain embodiments, the sterile saline is pharmaceutical grade PBS.

In certain embodiments, the double-stranded small interfering RNA compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

Pharmaceutical compositions comprising the double-stranded small interfering RNA compounds encompass any pharmaceutically acceptable salts, esters, or salts of such esters. In certain embodiments, pharmaceutical compositions comprising the double-stranded small interfering RNA compounds comprise one or more oligonucleotide which, upon administration to an animal, including a human, is capable of providing (directly or indirectly) the biologically active metabolite or residue thereof. Accordingly, for example, the disclosure is also drawn to pharmaceutically acceptable salts of the double-stranded small interfering RNA compounds, prodrugs, pharmaceutically acceptable salts of such prodrugs, and other bioequivalents.

Suitable pharmaceutically acceptable salts include, but are not limited to, sodium and potassium salts.

In certain embodiments, one or more the double-stranded small interfering RNA compounds provided herein is formulated as a prodrug. A prodrug can include the incorporation of additional nucleosides at one or both ends of a double-stranded small interfering RNA compound which are cleaved by endogenous nucleases within the body, to form the active antisense oligomeric compound. In certain embodiments, upon in vivo administration, a prodrug is chemically or enzymatically converted to the biologically, pharmaceutically or therapeutically more active form of an oligonucleotide. In certain embodiments, prodrugs are useful because they are easier to administer than the corresponding active form or to be processed by RISC. For example, in certain instances, a prodrug may be more bioavailable (e.g., through oral administration) than is the corresponding active form. In certain instances, a prodrug may have improved solubility compared to the corresponding active form. In certain embodiments, prodrugs are less water soluble than the corresponding active form. In certain instances, such prodrugs possess superior transmittal across cell membranes, where water solubility is detrimental to mobility.

In certain embodiments, pharmaceutical compositions provided herein comprise one or more the double-stranded small interfering RNA compounds and one or more excipients. In certain such embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

### Administration and Dosages

The siRNA molecules and the compositions comprising them may be administered to a subject by any suitable route. For example, the administration may be intravenous, subcutaneous, pulmonary, intramuscular, intraperitoneal, dermal, oral, nasal, or via inhalation.

For example, in certain embodiments, a pharmaceutical composition provided herein is prepared for oral administration. In certain embodiments, a pharmaceutical composition is prepared for administration by injection (e.g., intravenous, subcutaneous, intramuscular, dermal, intraperitoneal etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions.

In certain embodiments, a pharmaceutical composition is prepared for transmucosal administration. In some embodiments, penetrants appropriate to the barrier to be permeated are used in the formulation. Such penetrants are generally known in the art. In certain embodiments, a pharmaceutical composition is prepared for pulmonary delivery, e.g., intratracheal, intranasal or via inhalation. Compositions suitable for intranasal preparation can be administered into the nasal cavity as nasal suspensions. Compositions suitable for inhalation may be provided as pharmaceutical aerosols, for example, solution aerosols or powder aerosols, and may be administered using devices such as inhalers, e.g., metered dose inhalers (MDIs) or dry powder inhalers (DPIs), and nebulizers. By controlling the particle characteristics and deposition mechanics, or by specifically targeting certain cell types in the lung, for example by coupling ligands that bind to receptors expressed on the surface of target cells, compositions can be delivered to specific regions in the pulmonary tract. In certain embodiments, a pharmaceutical composition is prepared for intranasal administration. In certain embodiments it is administered via inhalation.

In certain embodiments, a pharmaceutical composition provided herein comprises the double-stranded small interfering RNA in a therapeutically effective amount. In certain embodiments, the therapeutically effective amount is sufficient to prevent, alleviate or ameliorate symptoms of a disease or to prolong the survival of the subject being treated. Determination of a therapeutically effective amount is well within the capability of those skilled in the art. In some embodiments, the administration comprises an effective dose of from 0.01 to 100 mg/kg. The administration may be once daily, weekly, every two weeks, monthly, every two months, or quarterly.

In certain embodiments, the present invention provides compositions and methods for reducing the amount or activity of a target nucleic acid in a cell. In certain embodiments, the cell is in an animal. In certain embodiments, the animal is a mammal. In certain embodiments, the animal is a rodent. In certain embodiments, the animal is a primate. In certain embodiments, the animal is a non-human primate. In certain embodiments, the animal is a human.

In certain embodiments, the present invention provides methods of administering a pharmaceutical composition comprising a double-stranded small interfering RNA compound of the present disclosure to an animal. Suitable administration routes include, but are not limited to, oral, rectal, transmucosal, intestinal, enteral, topical, suppository, through inhalation, intrathecal, intracerebroventricular, intraperitoneal, intranasal, intratumoral, and parenteral (e.g., intravenous, intramuscular, intramedullary, and subcutaneous). In certain embodiments, pharmaceutical intrathecals are administered to achieve local rather than systemic exposures. For example, pharmaceutical compositions may be injected directly in the area of desired effect (e.g., into the ears).

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH for the natural 2′-H of DNA) or as an RNA having a modified base (thymine (methylated uracil) for natural uracil of RNA).

Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” (SEQ ID NO: 67) encompasses any oligomeric compounds having such nucleobase sequence, whether modified or unmodified, including, but not limited to, such compounds comprising RNA bases, such as those having sequence “AUCGAUCG” (SEQ ID NO: 68) and those having some DNA bases and some RNA bases such as “AUCGATCG” (SEQ ID NO: 69) and oligomeric compounds having other modified or naturally occurring bases, such as “ATmeCGAUCG,” (SEQ ID NO: 70) wherein meC indicates a cytosine base comprising a methyl group at the 5-position.

Unless specific definitions are provided, the nomenclature utilized in connection with, and the procedures and techniques of, analytical chemistry, synthetic organic chemistry, and medicinal and pharmaceutical chemistry described herein are those well-known and commonly used in the art. Standard techniques may be used for chemical synthesis, and chemical analysis. Where permitted, all patents, applications, published applications and other publications, GENBANK Accession Numbers and associated sequence information obtainable through databases such as National Center for Biotechnology Information (NCBI) and other data referred to throughout in the disclosure herein are incorporated by reference for the portions of the document discussed herein, as well as in their entirety.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference for the portions of the document discussed herein, as well as in their entirety.

It is understood that the sequence set forth in each SEQ ID NO described herein is independent of any modification to a sugar moiety, an internucleoside linkage, or a nucleobase. As such, antisense compounds defined by a SEQ ID NO may comprise, independently, one or more modifications to a sugar moiety, an internucleoside linkage, or a nucleobase.

### EXAMPLES

The disclosure now being generally described, will be more readily understood by reference to the following examples, which are included merely for purposes of illustration of certain aspects and embodiments of the present disclosure, and is not intended to limit the disclosure.

### Example 1: Select siRNAs Targeting Human DUX4

16 siRNAs targeting human DUX4 were selected for assessment of DUX4 inhibition. The antisense and sense strand sequences of the selected siRNAs are provided in Table 1. Each siRNA listed in Table 1 is targeted against the human genomic DUX4 sequence (GENBANK Accession No. FJ439133.1, SEQ ID NO: 66).

TABLE 1
Sequences of Select Double-Stranded siRNAs Targeting Human DUX4

| | | | SEQ |
|---|---|---|---|
| Oligo Name | Strand | Nucleotide Sequence | ID NO |
| UGX1759 | Sense | CGUCCGUCCGUGAAAUUCC | 1 |
| | Antisense | GGAAUUUCACGGACGGACG | 2 |
| UGX1904 | Sense | GCUUUGAGCGGAACCCGUA | 3 |
| | Antisense | UACGGGUUCCGCUCAAAGC | 4 |
| UGX1979 | Sense | GGGUCCAGAUUUGGUUUCA | 5 |
| | Antisense | UGAAACCAAAUCUGGACCC | 6 |
| UGX1980 | Sense | GGUCCAGAUUUGGUUUCAG | 7 |
| | Antisense | CUGAAACCAAAUCUGGACC | 8 |
| UGX1982 | Sense | UCCAGAUUUGGUUUCAGAA | 9 |
| | Antisense | UUCUGAAACCAAAUCUGGA | 10 |
| UGX1983 | Sense | CCAGAUUUGGUUUCAGAAU | 11 |
| | Antisense | AUUCUGAAACCAAAUCUGG | 12 |
| UGX1984 | Sense | CAGAUUUGGUUUCAGAAUG | 13 |
| | Antisense | CAUUCUGAAACCAAAUCUG | 14 |
| UGX1985 | Sense | AGAUUUGGUUUCAGAAUGA | 15 |
| | Antisense | UCAUUCUGAAACCAAAUCU | 16 |
| UGX1986 | Sense | GAUUUGGUUUCAGAAUGAG | 17 |
| | Antisense | CUCAUUCUGAAACCAAAUC | 18 |
| UGX2204 | Sense | GGAUUCAGAUCUGGUUUCA | 19 |
| | Antisense | UGAAACCAGAUCUGAAUCC | 20 |
| UGX2211 | Sense | GAUCUGGUUUCAGAAUCGA | 21 |
| | Antisense | UCGAUUCUGAAACCAGAUC | 22 |
| UGX2212 | Sense | AUCUGGUUUCAGAAUCGAA | 23 |
| | Antisense | UUCGAUUCUGAAACCAGAU | 24 |
| UGX2951 | Sense | AGGCGCAACCUCUCCUAGA | 25 |
| | Antisense | UCUAGGAGAGGUUGCGCCU | 26 |
| UGX3033 | Sense | CGAGGAAGAAUACCGGGCU | 27 |
| | Antisense | AGCCCGGUAUUCUUCCUCG | 28 |
| UGX3050 | Sensc | CUCUGCUGGAGGAGCUUUA | 29 |
| | Antisense | UAAAGCUCCUCCAGCAGAG | 30 |
| UGX3241 | Sense | CGCGGUGUUCUGCCUUUCU | 31 |
| | Antisense | AGAAUGGCAGUUCUCCGCG | 32 |

### Example 2: Modifications of Select siRNAs Targeting Human DUX4

The select siRNAs shown in Table 1 were chemically modified to determine the effect of modification on the potency of the siRNAs. In short, the siRNAs were modified to incorporate a pattern of alternating 2′-O-Methy-modified/2′-Fluoro modified bases; all but one siRNA (UGX19.1) incorporate a locked nucleic acid (LNA) at the 5′ position on the sense strand (while UGX19.1 includes a 2′-Flouro (2′-F) modified G nucleoside at the 5′ position on the sense strand); all sense and antisense strands include phosphorothioate internucleoside linkages at the first and second positions (from the 5′ end); and all sense and antisense strands include two T nucleotides (TT overhangs) with phosphorothioate linkages at the 3′ end. The sequences of the siRNAs with modifications are shown in Table 2 (see the Table 2 Legend below for details about symbols and nomenclature used to identify modifications).

TABLE 2
Modifications of Select Double-Stranded siRNAs Targeting Human DUX4

| | | | SEQ |
|---|---|---|---|
| | | | ID |
| Oligo Name | Strand | Sequence with Modifications | NO |
| UGX1759.1 | Sense | +C*mG*fUmCfCmGfUmCfCmGfUmGfAmAfAmUfUmCfC*T*T | 33 |
| | Antisense | mG*fG*mAfAmUfUmUfCmAfCmGfGmAfCmGfGmAfCmG*T*T | 34 |
| UGX1904.1 | Sense | +G*mC*fUmUfUmGfAmGfCmGfGmAfAmCfCmCfGmUfA*T*T | 35 |
| | Antisense | mU*fA*mCfGmGfGmUfUmCfCmGfCmUfCmAfAmAfGmC*T*T | 36 |
| UGX1979.1 | Sense | +G*mG*fGmUfCmCfAmGfAmUfUmUfGmGfUmUfUmCfA*T*T | 37 |
| | Antisense | mU*fG*mAfAmAfCmCfAmAfAmUfCmUfGmGfAmCfCmC*T*T | 38 |
| UGX1980.1 | Sense | fG*mG*fUmCfCmAfGmAfUmUfUmGfGmUfUmUfCmAfG*T*T | 39 |
| | Antisense | mC*fU*mGfAmAfAmCfCmAfAmAfUmCfUmGfGmAfCmC*T*T | 40 |
| UGX1980.2 | Sense | +G*mG*fUmCfCmAfGmAfUmUfUmGfGmUfUmUfCmAfG*T*T | 41 |
| | Antisense | mC*fU*mGfAmAfAmCfCmAfAmAfUmCfUmGfGmAfCmC*T*T | 40 |
| UGX1982.1 | Sense | +U*mC*fCmAfGmAfUmUfUmGfGmUfUmUfCmAfGmAfA*T*T | 42 |
| | Antisense | mU*fU*mCfUmGfAmAfAmCfCmAfAmAfUmCfUmGfGmA*T*T | 43 |
| UGX1983.1 | Sense | +C*mC*fAmGfAmUfUmUfGmGfUmUfUmCfAmGfAmAfU*T*T | 44 |
| | Antisense | mA*fU*mUfCmUfGmAfAmAfCmCfAmAfAmUfCmUfGmG*T*T | 45 |
| UGX1984.1 | Sense | +C*mA*fGmAfUmUfUmGfGmUfUmUfCmAfGmAfAmUfG*T*T | 46 |
| | Antisense | mC*fA*mUfUmCfUmGfAmAfAmCfCmAfAmAfUmCfUmG*T*T | 47 |
| UGX1985.1 | Sense | +A*mG*fAmUfUmUfGmGfUmUfUmCfAmGfAmAfUmGfA*T*T | 48 |
| | Antisense | mU*fC*mAfUmUfCmUfGmAfAmAfCmCfAmAfAmUfCmU*T*T | 49 |
| UGX1986.1 | Sense | +G*mA*fUmUfUmGfGmUfUmUfCmAfGmAfAmUfGmAfG*T*T | 50 |
| | Antisense | mC*fU*mCfAmUfUmCfUmGfAmAfAmCfCmAfAmAfUmC*T*T | 51 |
| UGX2204.1 | Sense | +G*mG*fAmUfUmCfAmGfAmUfCmUfGmGfUmUfUmCfA*T*T | 52 |
| | Antisense | mU*fG*mAfAmAfCmCfAmGfAmUfCmUfGmAfAmUfCmC*T*T | 53 |
| UGX2211.1 | Sense | +G*mA*fUmCfUmGfGmUfUmUfCmAfGmAfAmUfCmGfA*T*T | 54 |
| | Antisense | mU*fC*mGfAmUfUmCfUmGfAmAfAmCfCmAfGmAfUmC*T*T | 55 |
| UGX2212.1 | Sense | +A*mU*fCmUfGmGfUmUfUmCfAmGfAmAfUmCfGmAfA*T*T | 56 |
| | Antisense | mU*fU*mCfGmAfUmUfCmUfGmAfAmAfCmCfAmGfAmU*T*T | 57 |
| UGX2951.1 | Sense | +A*mG*fGmCfGmCfAmAfCmCfUmCfUmCfCmUfAmGfA*T*T | 58 |
| | Antisense | mU*fC*mUfAmGfGmAfGmAfGmGfUmUfGmCfGmCfCmU*T*T | 59 |
| UGX3033.1 | Sense | +C*mG*fAmGfGmAfAmGfAmAfUmAfCmCfGmGfGmCfU*T*T | 60 |
| | Antisense | mA*fG*mCfCmCfGmGfUmAfUmUfCmUfUmCfCmUfCmG*T*T | 61 |
| UGX3050.1 | Sense | +C*mU*fCmUfGmCfUmGfGmAfGmGfAmGfCmUfUmUfA*T*T | 62 |
| | Antisense | mU*fA*mAfAmGfCmUfCmCfUmCfCmAfGmCfAmGfAmG*T*T | 63 |
| UGX3241.1 | Sense | +C*mG*fCmGfGmUfGmUfUmCfUmGfCmCfUmUfUmCfU*T*T | 64 |
| | Antisense | mA*fG*mAfAmUfGmGfCmAfGmUfUmCfUmCfCmGfCmG*T*T | 65 |

Table 2 Legend
+X = Locked Nucleic Acid (LNA) base, where X = A, U, C, or G
mX = 2′-O-Methyl (2′-OMe) modified RNA base X, where X = A, U, C, or G
fX = 2′-Flouro (2′-F) modified RNA base X, where X = A, U, C, or G
* = Phosphorothioated DNA (phosphorothioate bonds in these positions)

### Example 3: Potency of Select siRNAs Targeting Human DUX4

Properties such as tolerability, toxicity, efficacy, and stability of individual siRNAs cannot be predicted and must be empirically evaluated independently for each siRNA. Therefore, the siRNAs listed in Table 2 were tested for inhibition of DUX4 gene expression in vitro as described below.

Cells and Cell Culture. Immortalized human FSHD1 (54-2) (Krom et al., 2012) and FSHD2 (MB200) (Stadler et al., 2011) cell lines were used. Immortalized myoblasts were grown in Ham's F-10 Nutrient Mix (Gibco, Waltham, MA, USA) supplemented with 20% Corning USDA Approved Source Fetal Bovine Serum (Corning, Corning, NY, USA), 100 U/100 g penicillin/streptomycin (Gibco), 10 ng/ml recombinant human fibroblast growth factor (Promega Corporation, Madison, WI, USA) and 1 μM dexamethasone (Sigma-Aldrich). Differentiation of myoblasts into myotubes was achieved by switching confluent myoblast monolayers into DMEM:F-12 Nutrient Mixture (1:1, Gibco) supplemented with 2% KnockOut Serum Replacement (Gibco), 100 U/100 g penicillin/streptomycin, 10 μg/ml insulin and 10 μg/ml transferrin (KSR media) for 40 hours.

Small interfering RNA (siRNA) transfections. Modified duplex 21-mer siRNAs listed in Table 2 containing 19 base-pair complementary sequences and dTdT 3 prime overhangs were synthesized and obtained from Thermo Fisher Scientific or Integrated DNA Technologies. Transfections of siRNAs into FSHD1 and FSHD2 myotubes were carried out using Lipofectamine RNAiMAX (Invitrogen) according to the manufacturer's instructions. Briefly, cells were seeded at 1×10⁵ cells/well in 12-well plates and transfected approximately 20 hours later with 2 μL Lipofectamine RNAiMAX and 10 pmol or less of the selected siRNAs diluted in 100 μl Opti-MEM Reduced Serum Medium. 24, 48 or 72 hours following transfection, cells were incubated in differentiation medium for 40 hours to induce differentiation into myotubes and harvested for total RNA analysis.

Measurement of DUX4 target gene expression in FSHD1 and FSHD2 myotubes. Total RNA was extracted from whole cells using the RNeasy Mini Kit (Qiagen, Hilden, Germany) according to the manufacturer's instructions. Isolated RNA was treated with DNase I (Thermo Fisher Scientific), heat inactivated and reverse transcribed into cDNA using Superscript III (Thermo Fisher Scientific) and oligo(dT) primers (Invitrogen) following the manufacturer's protocol. qPCR was performed on cDNA to measure expression of DUX4. Expression of DUX4 can be measured by measuring expression of genes that are upregulated by DUX4, e.g., MBD3L2, ZSCAN4, LEUTX, MYOG, and MYH2 using TaqMan Gene Expression Assay ID numbers: MBD3L2, Hs00544743_ml; MYH2, Hs00430042_ml; MYOG, Hs01072232_ml; RPL30, Hs00265497_ml; LEUTX, Hs01028718_ml; ZSCAN4, Hs00537549_ml; or DUX4 with primers GCCGGCCCAGGTACCA (SEQ ID NO: 71) and CAGCGAGCTCCCTTGCA (SEQ ID NO: 72) with probe CAGTGCGCACCCCG (SEQ ID NO: 73) having a florescent dye (6FAM) attached at the 5′ end of the probe and a minor groove binder (MGB) and a non-florescent quencher (NFQ) attached at the 3′ end of the probe.

Evaluation of Potency of Select DUX4 siRNAs in FSHD1 and FSHD2 myotubes. To assess potency of siRNAs with modifications shown in Table 2, transfections of siRNAs into MB200 or 54-2 myoblasts were carried out using Lipofectamine RNAiMAX (Invitrogen) as above. Briefly, cells were seeded at 1×10⁵ cells/well in 1 ml regular growth medium in 12-well culture plates. Approximately 20 hours later, cells were transfected by first diluting 2 μl Lipofectamine RNAiMAX and 10, 2 or 0.4 pmol of either gene-specific siRNAs or a scrambled non-silencing control siRNA in 100 μl Opti-MEM Reduced Serum Medium to create lipofectamine/siRNA complexes. The lipofectamine/siRNA complexes were then added dropwise to one well of the culture plate containing myoblasts. 24 hours following transfection, cells were incubated in differentiation medium for 40 hours to induce differentiation into myotubes and harvested for total RNA analysis.

For determination of the 5000 inhibitory concentration (IC50) for each siRNA, seven-point concentration response curves were generated by first creating 3-fold serial dilutions of siRNAs from concentrated stocks in water in 96-well plates. Transfection then proceeded as above. IC50s were determined by nonlinear regression using a four-parameter logistic equation (GraphPad Prism Software Inc., San Diego, CA). Data is presented as IC50s with two significant digits. Data from the IC50 studies are provided in Table 3, where activity of DUX4 is reported on the basis of MBD3L2 expression.

TABLE 3
Potency of Select Modified siRNAs

IC50 (nM)

| Oligo Name | MB200 | 54-2 |
|---|---|---|
| UGX1759.1 | 3.1 | |
| UGX1904.1 | † 25% incr. | † 10% incr. |
| UGX1979.1 | 0.97 | 3.6 |
| UGX1980.1 | 1.5 | 0.71 |
| UGX1980.1 | 1.6 | 2.9 |
| UGX1982.1 | 4.6 | 1.8 |
| UGX1983.1 | NA | † 73% incr. |
| UGX1984.1 | 0.29 | 0.12 |
| UGX1985.1 | 2.81 | 3.37 |
| UGX1986.1 | 42 | NA |
| UGX2204.1 | 0.34 | 2.5 |
| UGX2211.1 | 1.8 | 4.6 |
| UGX2212.1 | 0.25 | 1.1 |
| UGX2951.1 | † 70% incr. | † 98% incr. |
| UGX3033.1 | NA | † 19% incr. |
| UGX3050.1 | † 24% incr. | † 20% incr. |
| UGX3241.1 | 6.9 | NA |

In Table 3, “†” indicates an increase in DUX4 activity was observed at the 10 nM siRNA dose (i.e., DUX4 activity increased by 25% with 10 nM UGX1904.1; 70% with 10 nM UGX2951.1; and 24% with 10 nM UGX3050.1).

As shown in Table 4, three siRNAs yielded sub-nanomolar IC50s in MB200 cells: UGX1984.1, UGX2204.1, and UGX2212.1. Surprisingly, the modification of these three highly effective siRNAs resulted in minimal loss of efficacy as compared to siRNAs of the same base sequence but without the modifications (i.e., as compared to naked siRNA oligonucleotides of the same sequence). Table 4 shows the IC50 (nM) of each of these three siRNAs in modified and non-modified form. In Table 4, “fully modified” means the alternating 2′-O-Methy-modified/2′-Fluoro modified bases, a locked nucleic acid (LNA) at the 5′ position on the sense strand, phosphorothioate internucleoside linkages at the first and second positions from the 5′ end on all sense and antisense strands, and two T nucleotides (TT overhangs) with phosphorothioate linkages at the 3′ end on all sense and antisense strands (as shown in Table 2), and “no modifications” means naked siRNA oligonucleotides of the same sequence (i.e., of the corresponding sequence provided in Table 1).

TABLE 4
Minimal Loss of Efficacy in Fully Modified
Select Sub-Nanomolar siRNAs

| Oligo | Modification | IC50 in MB200 | IC50 in 54-2 |
|---|---|---|---|
| name | pattern | cells (nM) | cells (nM) |
| UGX1984 | no modifications | 0.28 | 0.23 |
| UGX1984.1 | fully modified | 0.29 | 0.12 |
| UGX2204 | no modifications | 0.21 | 0.37 |
| UGX2204.1 | fully modified | 0.34 | 2.5 |
| UGX2212 | no modification | 0.18 | 0.04 |
| UGX2212.1 | fully modified | 0.25 | 1.1 |

### Example 4: Further Evaluation of DUX4 siRNA 1984.1 Potency

Fully modified DUX4 siRNA 1984.1 appeared to be highly efficacious with subnanomolar IC50 calculated in both MB200 and 54-2 myotubes based on MBD3L2 expression. To further evaluate the efficacy of this double-stranded siRNA, IC50s were calculated in MB200 and 54-2 myotubes based on DUX4 expression itself, as well as additional DUX4 targets ZSCAN and LEUlX. Briefly, MB200 (FSHD type 2) and 54-2 (FSHD type 1) myoblasts were transfected with negative control siRNA or fully modified double-stranded siRNA UGX1984.1 using RNAiMax. Cells were differentiated the following day with Differentiation Media and total RNA harvested at 40 hr. DUX4 target genes were assessed with qPCR using standard TaqMan reagents with RPL30 as an internal control and DUX4 transcript was determined using a custom TaqMan according to Example 3 above with RPL30 as an internal control. Results are provided in Table 5.

TABLE 5
Further evaluation of double-stranded siRNA UGX1984.1

| Target Gene | IC50 in MB200 cells (nM) | IC50 in 54-2 cells (nM) |
|---|---|---|
| DUX4 | 0.47 | 0.29 |
| ZSCAN | 0.16 | 0.12 |
| LEUTX | 0.17 | 0.20 |
| MBD3L2 | 0.29 | 0.12 |

All publications, patents and literature specifically mentioned herein are incorporated by reference in their entireties for all purposes.

1. A double-stranded small interfering RNA (siRNA) comprising a sense strand and an antisense strand, wherein the antisense strand comprises a nucleobase sequence of at least 12 contiguous nucleotides of a sequence selected from SEQ ID NOs: 14, 20, 24, 2, 4, 6, 8, 10, 12, 16, 18, 22, 26, 28, 30, and 32, and wherein the double-stranded siRNA comprises one or more of: (i) alternating 2′-O-Methyl-modified/2′-Fluoro-modified bases in the sense and/or antisense strands; (ii) a locked nucleic acid (LNA) at the 5′ position of the sense strand or a 2′-Flouro (2′-F) modified G nucleoside at the 5′ position of the sense strand; and (iii) phosphorothioate internucleoside linkages between the first and second bases and between the second and third bases from the 5′ end of the sense and antisense strands.

2-4. (canceled)

5. The double-stranded siRNA of claim 1, wherein the antisense strand comprises a TT overhang at the 3′ end.

6. The double-stranded siRNA of claim 1, wherein the sense strand comprises a TT overhang at the 3′ end.

7-9. (canceled)

10. The double-stranded siRNA of claim 1, wherein the siRNA is conjugated to a lipophilic molecule, an antibody, an aptamer, a ligand, a peptide, or a polymer.

11. The double-stranded siRNA of claim 10, wherein the lipophilic molecule is a long chain fatty acid (LCFA).

12. The double-stranded siRNA of claim 10, wherein the antibody is an anti-transferrin receptor antibody.

13. A pharmaceutical composition comprising the double-stranded siRNA of claim 1 and a pharmaceutically acceptable carrier.

14-18. (canceled)

19. A method for ameliorating, preventing, delaying onset of, or treating a disease or disorder associated with aberrant expression of DUX4 in a subject in need thereof, the method comprising administering to the subject the pharmaceutical composition of claim 13.

20. The method of claim 19, wherein the disease or disorder is FSHD.

21. The method of claim 20, wherein the FSHD is selected from the group consisting of FSHD1 and FSHD2.

22-28. (canceled)

29. The double-stranded siRNA of claim 1, wherein the siRNA is UGX1984.1 comprising a sense strand nucleotide sequence according to SEQ ID NO: 46 and an antisense strand nucleotide sequence according to SEQ ID NO: 47.

30. The double-stranded siRNA of claim 1, wherein the siRNA is UGX2204.1 comprising a sense strand nucleotide sequence according to SEQ ID NO: 52 and an antisense strand nucleotide sequence according to SEQ ID NO: 53.

31. The double-stranded siRNA of claim 1, wherein the siRNA is UGX2212.1 comprising a sense strand nucleotide sequence according to SEQ ID NO: 56 and an antisense strand nucleotide sequence according to SEQ ID NO: 56.
